SEQUENCE LISTING (18 sequences)

SEQ ID NO: 1 - DNA, 12 bp, Artificial Sequence
  Description of Artificial Sequence Synthetic oligonucleotide
agcataaaaggc

SEQ ID NO: 2 - DNA, 26 bp, Artificial Sequence
  Description of Artificial Sequence Synthetic oligonucleotide
agatgaacggtgtacagaccaggcgc

SEQ ID NO: 3 - DNA, 29 bp, Artificial Sequence
  Description of Artificial Sequence Synthetic oligonucleotide
atggaaagcgcagtctctgaatttaccgt

SEQ ID NO: 4 - DNA, 38 bp, Artificial Sequence
  Description of Artificial Sequence Synthetic oligonucleotide
gtgtacactgactgactgactgactgactgtctctgaa

SEQ ID NO: 5 - DNA, 24 bp, Artificial Sequence
  Description of Artificial Sequence Synthetic oligonucleotide
cagtcagtcagtcagtcagtcagt

SEQ ID NO: 6 - DNA, 43 bp, Artificial Sequence
  Description of Artificial Sequence Synthetic oligonucleotide
nnnnngnnnnnnnnnnnnnnnggtaccnctcttcntgactgac

SEQ ID NO: 7 - DNA, 29 bp, Artificial Sequence
  Description of Artificial Sequence Synthetic oligonucleotide
ntaagagnggtaccnnnnnnnnnnnnnnn

SEQ ID NO: 8 - DNA, 37 bp, Artificial Sequence
  Description of Artificial Sequence Synthetic oligonucleotide
gtcagtcantaagagnggtaccnnnnnnnnnnnnnnn

SEQ ID NO: 9 - DNA, 26 bp, Artificial Sequence
  Description of Artificial Sequence Synthetic oligonucleotide
nnnnngnnnnnnnnnnnnnnnggtac

SEQ ID NO: 10 - DNA, 14 bp, Artificial Sequence
  Description of Artificial Sequence Synthetic oligonucleotide
antaagagnggtac

SEQ ID NO: 11 - DNA, 35 bp, Artificial Sequence
  Description of Artificial Sequence Synthetic oligonucleotide
ntaagagnggtaccnnnnnnnnnnnnnnngnnnnn

SEQ ID NO: 12 - DNA, 43 bp, Artificial Sequence
  Description of Artificial Sequence Synthetic oligonucleotide
gtcagtcantaagagnggtaccnnnnnnnnnnnnnnngnnnnn

SEQ ID NO: 13 - DNA, 17 bp, Artificial Sequence
  Description of Artificial Sequence Synthetic oligonucleotide
cgtgcgaattccctggc

SEQ ID NO: 14 - DNA, 17 bp, Artificial Sequence
  Description of Artificial Sequence Synthetic oligonucleotide
cgtgcuuuuuucctggc

SEQ ID NO: 15 - DNA, 17 bp, Artificial Sequence
  Description of Artificial Sequence Synthetic oligonucleotide
cgtgcagcagtcctggc

SEQ ID NO: 16 - DNA, 91 bp, Artificial Sequence
  Description of Artificial Sequence Synthetic oligonucleotide
actgactgactgactgactgactgactgactgactgactgactgactgactgactgactggatttttttttttctagcttaagctgcacgc

SEQ ID NO: 17 - DNA, 46 bp, Artificial Sequence
  Description of Artificial Sequence Synthetic oligonucleotide
cagtcagtccatccgcacggtcgaccgtgccgtcttctgcgcgtgc

SEQ ID NO: 18 - DNA, 32 bp, Artificial Sequence
  Description of Artificial Sequence Synthetic oligonucleotide
gcagaagacggcacggtcgaccgtgcggatgg

(19) United States
(12) Patent Application Publication
(10) Pub. No.: US 2012/0316075 A1
(43) Pub. Date: Dec. 13, 2012

(54) SEQUENCE PRESERVED DNA CONVERSION FOR OPTICAL NANOPORE SEQUENCING

(75) Inventors: Philip R. BUZBY, Brockton, MA (US); Amit MELLER, Brookline, MA (US); Benjamin MCNALLY, Brighton, MA (US); Andy FAN, Cambridge, MA (US); Edyta OLEJNIK-KRZYNMANSKA, Brookline, MA (US)

(73) Assignee: NOBLEGEN BIOSCIENCES, INC., Concord, MA (US)

(21) Appl. No.: 13/435,819

(22) Filed: Mar. 30, 2012

Related U.S. Application Data
(60) Provisional application No. 61/469,336, filed on Mar. 30, 2011.

(51) Int. Cl. C12P 19/34; C40B 20/08; B82Y 15/00

(52) U.S. Cl. 506/6; 435/91.53; 977/781; 977/902

(57) ABSTRACT

The present invention relates to a method for conversion of a target nucleic acid molecule according to a predetermined nucleotide code into a converted nucleic acid molecule. The converted nucleic acid molecule has utility for determining the nucleotide sequence of the target nucleic acid molecule, for example, using a nanopore.

### CROSS-REFERENCE TO RELATED APPLICATIONS

This application claims the benefit or priority to U.S. Provisional Application No. 61/469,336, filed Mar. 30, 2011, the entire disclosure of which is hereby incorporated by reference in its entirety.

### GOVERNMENT FUNDING

Some portion of this invention may have been funded by United States government grants NHGR11RO1-HG-004128, 1RO1-HG-005871, and R43HG006212. Therefore, the government may have some rights in this invention.

### FIELD OF THE INVENTION

The present invention relates to a method for conversion of a target nucleic acid molecule according to a predetermined nucleotide code into a converted nucleic acid molecule. The converted nucleic acid molecule has utility for determining the nucleotide sequence of the target nucleic acid molecule using a nanopore.

The instant application contains a Sequence Listing which has been submitted in ASCII format via EFS-Web and is hereby incorporated by reference in its entirety. Said ASCII copy, created on Aug. 2, 2012, is named 22613US2.txt and is 6,389 bytes in size.

### BACKGROUND

The pioneering completion of the 1^{st} reference human genome sequence (International Human Genome Sequencing Consortium Nature 2001; 490:860-921; Venter et al., Science 2001; 291:1304-51) has marked the commencement of an era in which genomic variations directly impact drug discovery and medical therapy. This new paradigm has created a need for inexpensive and ultra-fast methods for DNA sequencing. It is thought that in the near future, medical practitioners will be able to routinely analyze the DNA of individual patients in a clinical setting before prescribing drugs. Sequence information obtained from the individual could be checked against online databases in which genomic information relevant to any drug is documented.

In addition, affordable sequencing technologies will transform research in comparative genomics and molecular biology, allowing scientists to quickly sequence whole genomes from cell variants. To realize ultra-fast and inexpensive DNA sequencing, revolutionary technologies are needed to replace the classical methods based on Sanger's “dideoxy” protocol (Shendure et al., Nat Rev Genet 2004; 5:335-44). Modern sequencing based on the Sanger method typically produces a sequence that has poor quality in the first 15-40 bases, a high quality region of no more than 700-900 bases, and then quickly deteriorating quality for the remainder of the sequence.

New sequencing technologies need to address two major issues. First, sample size should be reduced to a minimum, enabling sequence readout from a single DNA molecule or a small number of copies. Second, readout speed should be increased by several orders of magnitude compared to current state-of-the-art techniques. In recent years, nanopores have been used extensively as sensitive single-biomolecule detectors. It has been shown that single-stranded DNA molecules can be electrophoretically driven through a 1.5-nm α-hemolysin nanopore in a single file manner. This process is termed DNA translocation (Kasianowicz et al., Proc Natl Acad Sci USA 1996; 93:13770-3; Akeson et al., Biophys J 1999; 77:3227-33; Meller et al., Proc Natl Acad Sci USA 2000; 97:1079-84). One of the driving ideas in this field has been that nanopores could be used for direct electronic readout of the DNA sequence (Deamer et al., Tibtech 2000; 18:147-50). Early studies, however, have indicated that several prominent issues must be addressed before nanopores can be used for single-molecule sequencing (Meller et al., (2000), supra; Meller et al., Phys Rev Lett 2001; 86:3435-8). In particular, fast DNA translocation speed and low contrast between the electrical signals of the 4 base types have prevented single nucleotide differentiation.

A major advantage of nanopore sequencing is that a single molecule of DNA can be probed directly using a nanopore, without the need for amplification of a DNA molecule, which is error-prone, low-throughput and costly. At present however, nanopore sequencing techniques do not have single nucleotide resolution. Although much progress has been made, the minimal number of bases that can be resolved by a nanopore has not been firmly established. Our approach has been to convert nucleic acid sequences into a longer sequence that can be converted so that the sequence is preserved. The longer sequence can then be read by a nanopore directly. Thus, the manner in which the conversion is done must be fast, highly reliable, inexpensive and permit efficient, facile recovery of the conversion product and there is a need to develop new methods for carrying out such conversions.

### SUMMARY OF THE INVENTION

The invention is based, in part, on the surprising discovery that a converted molecule can be accurately generated without introducing errors commonly associated with amplification processes such as PCR. Error-free cyclical production of the converted molecule, reading the sequence on a nanopore, and interpretation of the sequence is the basis of diagnosing genetic disease.

It is understood that any of the embodiments described below can be combined in any desired way, and any embodiment or combination of embodiments can be applied to each of the aspects described below, unless clearly exclusive.

In one aspect, the invention provides a method for conversion of a target nucleic acid comprising: (a) attaching a sample modifier comprising a moiety for immobilization to a solid support and a pre-specified sequence to a target nucleic acid, to provide a modified nucleic acid; (b) immobilizing the modified nucleic acid onto a solid support; (c) ligating a probe library comprising a pre-determined expanded code for one or more bases to the immobilized modified nucleic acid; (d) forming a circular molecule by circularizing the molecule produced in step (c); (e) cleaving the circular molecule with one or more restriction enzymes; and (f) repeating steps (c) to (e) two or more times, wherein the method results in a converted molecule which can be used to determine the nucleotide sequence of the target nucleic acid.

In some embodiments, the target nucleic acid is DNA or RNA.

In some embodiments, the moiety for immobilization to a solid support is a biotin moiety.

In some embodiments, the sample modifier is attached to the target nucleic acid using a ligase.

In some embodiments, the sample modifier comprises a barcode, a cleavage site, or a tag sequence.

In some embodiments, the cleavage site is a substrate for an enzyme or a chemical.

In some embodiments, the barcode identifies the sample of origin and is formed by the arrangements of the pre-determined expanded base codes forming a barcode 4-10 codes in length.

In some embodiments, the tag sequence identifies the 5′ end or the 3′ end of the converted molecule.

In some embodiments, the solid support is a magnetic particle, polymeric microsphere, or a filter material.

In some embodiments, the probe library comprises a plurality of distinct oligonucleotide sequences, each of which includes a double-stranded region, wherein the double-stranded region comprises: two restriction enzyme binding sites, the pre-specified nucleotide sequence, one or more pre-determined codes for each of the bases found in the target nucleic acid, and a first and a second single-stranded overhang, wherein the first single-stranded overhang is a complement to the pre-specified nucleotide sequence and the second single-stranded overhang comprises a plurality of sequences able to complement the target nucleic sequence.

In some embodiments, the pre-determined base codes for each base bind to a molecular beacon.

In some embodiments, there are four pre-determined base codes.

In some embodiments, forming the circular molecule comprises use of a ligase.

In some embodiments, the ligase is a DNA ligase or an RNA ligase.

In some embodiments, forming the circular molecule comprises removal of a blocker molecule from the probe library.

In some embodiments, the blocker molecule comprises DNA, RNA, PNA, or LNA.

In some embodiments, the restriction enzyme is a Type IIs restriction enzyme.

In some embodiments, a first restriction enzyme is a Type II restriction enzyme and a second restriction enzyme is Type IIs restriction enzyme.

In some embodiments, cleavage with the Type II restriction enzyme and the Type IIs restriction enzyme is performed in a single step.

In some embodiments, the Type IIs restriction enzyme cleaves 1 to 4 bases from the end of the target nucleic acid.

In another aspect, the invention provides a method for sequencing of a target nucleic acid comprising: (a) attaching a sample modifier comprising a moiety for immobilization to a solid support and a pre-specified sequence to a target nucleic acid to provide a modified nucleic acid; (b) immobilizing the modified nucleic acid onto a solid support; (c) ligating a probe library comprising a pre-determined expanded code for one or more bases to the immobilized modified nucleic acid; (d) forming a circular molecule by circularizing the molecule produced in step (c); (e) cleaving the circular molecule with one or more restriction enzymes; (f) repeating steps (c) to (e) two or more times to provide a converted molecule; (g) hybridizing the converted molecule to a plurality of detectably labeled molecules to form a complex; (h) detaching the complex from the solid support; and (i) translocating the complex through a nanopore, wherein the translocation produces detectable signals which can be used to determine the nucleotide sequence of the target nucleic acid.

In some embodiments, the target nucleic acid is DNA or RNA.

In some embodiments, the moiety for immobilization to a solid support is a biotin moiety.

In some embodiments, the sample modifier is attached to the target nucleic acid using a ligase.

In some embodiments, the sample modifier comprises a barcode, a cleavage site, or a tag sequence.

In some embodiments, the cleavage site is a substrate for an enzyme or a chemical.

In some embodiments, the barcode identifies the sample of origin and is formed by the arrangement of the pre-determined expanded base codes forming a barcode 4-10 codes in length.

In some embodiments, the tag sequence identifies the 5′ end or the 3′ end of the converted molecule.

In some embodiments, the solid support is a magnetic particle, polymeric microsphere, or a filter material.

In some embodiments, the probe library comprises a plurality of distinct oligonucleotide sequences, each of which includes a double-stranded region, wherein the double-stranded region comprises: two restriction enzyme binding sites, the pre-specified nucleotide sequence, one or more pre-determined codes for each of the bases found in the target nucleic acid, and a first and a second single-stranded overhang, wherein the first single-stranded overhang is a complement to the pre-specified nucleotide sequence and the second single-stranded overhang comprises a plurality of sequences able to complement the target nucleic sequence.

In some embodiments, the pre-determined base codes bind to a molecular beacon.

In some embodiments, there are four pre-determined base codes.

In some embodiments, forming the circular molecule comprises use of a ligase.

In some embodiments, the ligase is a DNA ligase or an RNA ligase.

In some embodiments, forming the circular molecules comprises removal of a blocker molecule from the probe library.

In some embodiments, the blocker molecule comprises DNA, RNA, PNA, or LNA.

In some embodiments, the restriction enzyme is a Type IIs restriction enzyme.

In some embodiments, a first restriction enzyme is a Type II restriction enzyme and a second restriction enzyme is Type IIs restriction enzyme.

In some embodiments, the Type II and Type IIs restriction enzymes are combined together in a single step.

In some embodiments, the Type IIs restriction enzyme cleaves 1 to 4 bases from the end of the target nucleic acid.

In some embodiments, the detectably labeled molecules are optically detectable.

In some embodiments, the detectably labeled molecules comprise a fluorophore.

In some embodiments, the detectably labeled molecules comprise a fluorophore and a quencher.

In some embodiments, the detectably labeled molecules comprise a bulky group.

In some embodiments, the step of detaching from the solid support comprises using light, a chemical, or an enzyme.

In some embodiments, the enzyme is a restriction enzyme.

In some embodiments, the chemical is silver or periodate.

### BRIEF DESCRIPTION OF THE FIGURES

FIG. 1A illustrates the process of attaching a sample modifier to the target nucleic acid, specifically to fragmented dsDNA; the sample modifier provides a moiety for surface attachment and a pre-specified sequence which is the initiation for the conversion process.

FIG. 1B illustrates various configurations of the sample modifier that include a labile chemical linkage for release from the surface following conversion and a barcode for sample tracking.

FIG. 2 illustrates the conversion process for a single molecule with the target sequence AGCATAAAAGGC (SEQ ID NO: 1) being converted, hybridized with detectable probes, and finally released from the surface for subsequent analysis to determine original target sequence.

FIGS. 3A-3E illustrate the conversion cycle process using single base/cycle code expansion.

FIG. 3A illustrates the probe library hybridized to target single-stranded nucleic acid previously modified with the sample modifier and attached to a solid support. FIG. 3A discloses SEQ ID NOS 6-7, respectively, in order of appearance.

FIG. 3B illustrates one end of probe library ligated to the interrogation end of the target nucleic acid, the end for which the base will be expanded. FIG. 3B discloses SEQ ID NOS 6 and 8, respectively, in order of appearance.

FIG. 3C illustrates the other end of the probe library ligated to the pre-specified sequence (the sample modifier) forming the circular intermediate. FIG. 3C discloses SEQ ID NOS 6 and 8, respectively, in order of appearance.

FIG. 3D illustrates the circular molecule cleaved using restriction enzymes removing a single base from the end of the target nucleic. FIG. 3D discloses SEQ ID NOS 9-10, respectively, in order of appearance.

FIG. 3E illustrates the sample treated to make single-stranded and ready for next conversion cycle (in FIG. 2, 12 of these conversion cycles would have been performed to generate the expand converted molecule shown).

FIG. 4A-4E illustrates the conversion cycle process when using a blocker molecule and single base/cycle code expansion. The blocker molecule prevents ligation of the end comprising the pre-specified sequence until after the probe library has been joined at the interrogation end.

FIG. 4A illustrates the probe library with the block molecule hybridized to a target single-stranded nucleic acid previously modified with the sample modifier and attached to a solid support. FIG. 4A discloses SEQ ID NOS 6 and 11, respectively, in order of appearance.

FIG. 4B illustrates one end of probe library ligated to the interrogation end of the target nucleic acid, the end for which the base will be expanded. FIG. 4B discloses SEQ ID NOS 6 and 12, respectively, in order of appearance.

FIG. 4C illustrates the blocker molecule removed, allowing the other end of the probe library to be ligated to the pre-specified sequence (the sample modifier) forming the circular intermediate. FIG. 4C discloses SEQ ID NOS 6 and 8, respectively, in order of appearance.

FIG. 4D illustrates the circular molecule cleaved using restriction enzymes removing a single base from the end of the target nucleic acid. FIG. 4D discloses SEQ ID NOS 9-10, respectively, in order of appearance.

FIG. 4E illustrates the sample treated to make single-stranded and ready for next conversion cycle.

FIG. 5A illustrates a probe library designed to expand a single base code per conversion cycle, specifically a probe library molecule for single based coding. There is a single pre-determined code for the terminal base on the target nucleic acid (X/x′). The Type IIs restriction enzyme cleaves a single base into the target nucleic acid. It is not required that the Type IIs have a 1 base cleavage specificity, as shown the probe library may be modified with additional bases (blank boxes) to space the cleavage site in the desired location.

FIG. 5B illustrates a probe library modified to convert 4 bases (w′/x′/y′/z′) per conversion cycle by inclusion of 4 pre-determined codes (w′/x′/y′/z′) and positioning of the Type IIs cleavage site to remove 4 bases per conversion cycle from the target nucleic acid, specifically a probe library molecule for mult-based coding.

FIG. 6A-6E illustrates a sample modifier that may contain a cleavage site to permit release from the solid surface.

FIG. 6A illustrates that a restriction enzyme site which when made double-stranded and incubated with the enzyme EcoRI cleaves the converted molecule from the surface. FIG. 6A discloses SEQ ID NO: 13.

FIG. 6B illustrates that various nucleoside bases may be included singly or in multiples producing many cleavage sites only one of which is required to cleave. Cleavage of dU is enzymatic using 2 enzymes: Uracil DNA glycosylase (UDG) and the DNA glycosylase-lyase Endonuclease VIII. FIG. 6B discloses SEQ ID NO: 14.

FIG. 6C illustrates that the backbone of the nucleic acid of the sample modifier might include thiophosphate(s) which are cleavable by treatment with silver. FIG. 6C discloses SEQ ID NO: 15.

FIG. 6D illustrates that the sample modified might include a nonnucleoside component containing the labile chemical linkage, the specific example shown is a linker cleavable by exposure to light (see Piggott et al. 2005; Tettrahedron Letters 46:8241-8244).

FIG. 6E illustrates a linker comprising a vicinal diol (may be either nonnucleosidic or one or more RNA residues) which upon exposure to periodate results in a cleavage.

FIG. 7A illustrates sample barcoding using the expanded code configuration, i.e., an arrangement of a barcode on the sample modifier using the 4 pre-determined codes for base w′, x′, y′, and z′. Shown is the order w′/x′/y′/z′ which might be used for a single sample, however every unique arrangement of w′, x′, y′, z′ from 4-10 codes in length are possible.

FIG. 7B illustrates an arrangement for a barcode using a sequence of individual bases configuration, i.e., a unique arrangements of nucleoside bases (N) which must undergo the conversion process to expanded codes to be read.

FIG. 8 illustrates an example of a single-stranded target DNA (template) and a probe library (1 of 4) used in the conversion process to generate the images shown in FIG. 9A and FIG. 9B. The 3 probe libraries not shown had the template binding sequences: GACTGACT, ACTGACTG, TGCATGCA. The 4 probes libraries were added as a mixture. The template has all the elements for the conversion process and surface release. The sequence is repeating units of “ACTG” which hybridize specifically with each of the 4 libraries. The restriction enzymes coded for in the probe library were SalI and BtsCI with the BtsCI positioned to remove a single base per conversion cycle from the template. FIG. 8 discloses SEQ ID NOS 16-18, respectively, in order of appearance.

FIG. 9A is a graphic representation of a polyacrylamide gel image showing the sequential analysis of 8 cycles of conversion at the step of forming circular molecules. Only the expected largest circles are highlighted however since reactions are not 100%, within each lane bands from circle C-1, C-2, C-3, etc. may be seen.

FIG. 9B is a graphic representation of a polyacrylamide gel image showing the sequential analysis of 8 cycles of conversion at the step following Type II SalI/Type IIs BtsCI restriction enzyme cleavage. As with the gel in FIG. 9A, only the longest linear product is highlighted and similar to image in FIG. 9A within each lane bands from C-1, C-2, C-3, etc. may be seen.

FIGS. 10A and 10B illustrate a model system for preparing a kilobase or larger circular ssDNA as desirable using the described conversion process when the length of fragmented nucleic acid coupled with the expanded conversion exceed 1,000 bases. The bacteriophage M13 DNA is single-stranded, circular, ˜7,420 b in length and available commercially. As shown, it is possible to define various restriction enzymes sites (restriction enzyme 1, restriction enzyme 2) at targeted locations around the DNA. Cleavage by the 2 restriction enzymes followed by treatment with a phosphatase enzyme (SAP) to remove terminal phosphates produces fragments of various lengths incapable of re-joining. In the example, restriction enzyme 1=BsrGI and restriction enzyme 2=AlwNI, which generate fragments approx 6,000 (top band lane 4, FIG. 10B) and 1,000 (lower band lane 4, FIG. 10B) bases in length. The “Spanner 1” mimics features of both the probe library (code expansion) and sample modifier (moiety for surface attachment). The single-stranded ends of the Spanner are designed to join a specific fragment in the M13 digestion, in the example specifically the ˜1,000 b fragment, forming a ˜1 kB circle (band in lanes 8 and 10, FIG. 10B).

### DETAILED DESCRIPTION OF THE INVENTION

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

### Definitions

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≧0 and ≦2 if the variable is inherently continuous.

As used herein, unless specifically indicated otherwise, the word “or” is used in the inclusive sense of “and/or” and not the exclusive sense of “either/or.”

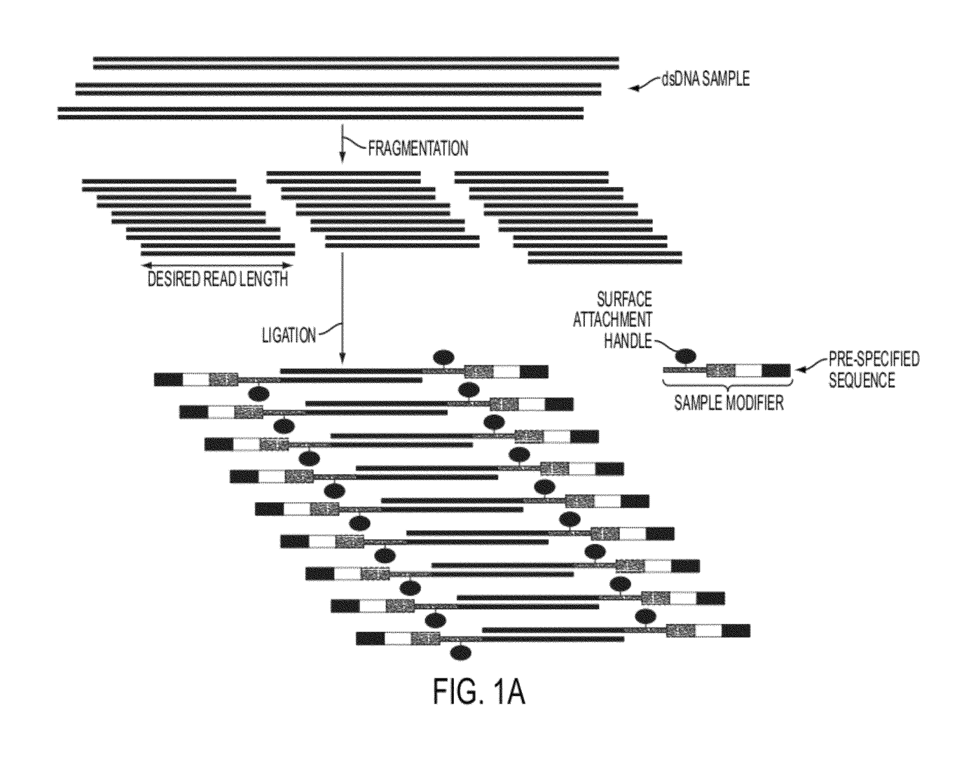

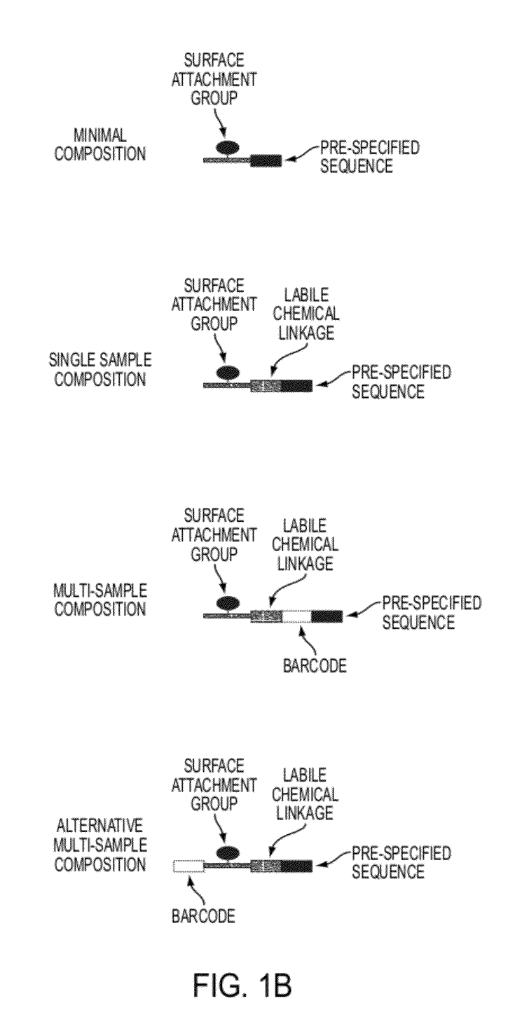

As used herein, the term “conversion” is used to describe the process of substituting an oligonucleotide code to represent or encode a given nucleotide, for example such that the code can be used for further sequencing and thus it is not necessary for the sequencing method to read at the single nucleotide level (see FIG. 1). The term “conversion” is also intended to encompass conversion of more than one nucleotide at a time (e.g., at least 2, at least 3, at least 4, at least 5, at least 6, or more nucleotides to be converted at one time). The term “converted ssDNA”, “converted target ssDNA”, “converted target” or “synthetic representation” is used to describe a DNA (or RNA) molecule that has undergone at least one round of conversion. The oligonucleotide code used as a representative of each given nucleotide in a converted ssDNA is also referred to herein as a “predetermined oligonucleotide code”, which can comprise a binary code as described in the Detailed Description herein.

As used herein, the terms “probe” and “oligonucleotide probe” are used to refer to an oligonucleotide produced synthetically, which is capable of annealing with or specifically hybridizing to a nucleic acid that comprises a sequence complementary to the probe. The exact length of the probe will depend upon many factors, including temperature, restriction enzymes used, number of copies of each probe in a probe library and the method used. An oligonucleotide probe, for use in the methods described herein for conversion, comprises a double-stranded portion with two flanking single-stranded overhangs each on both ends of one strand. Such probes are also referred to herein as “conversion probes.”

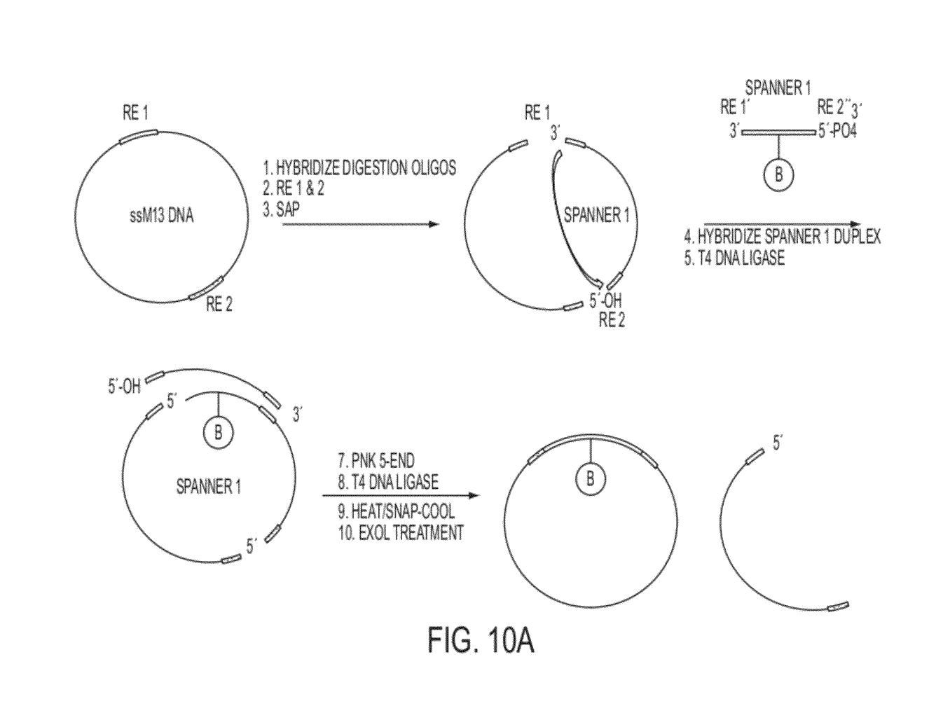

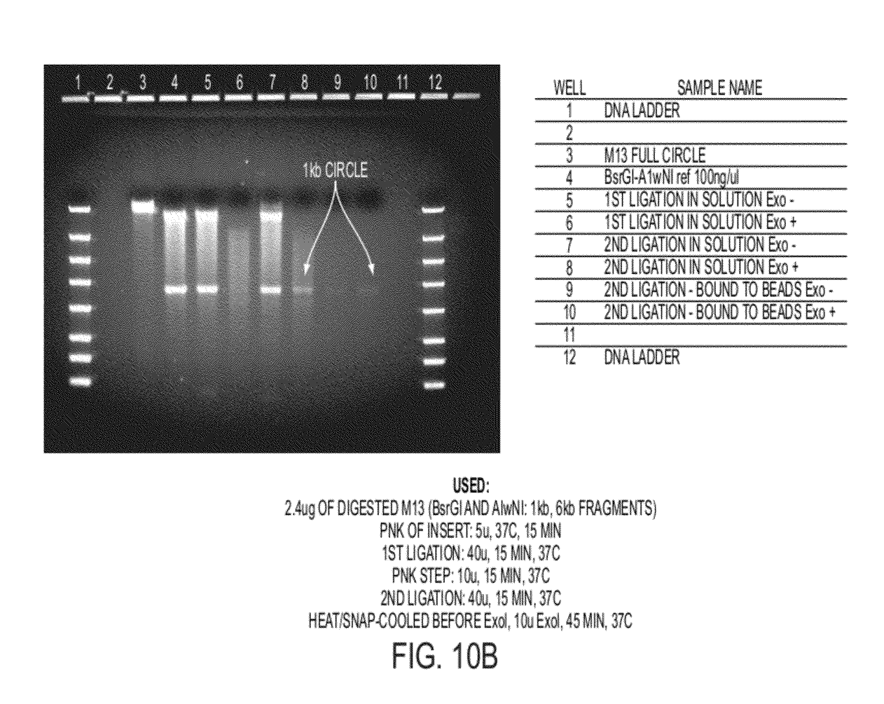

As used herein, the term “probe library” refers to a plurality of distinct oligonucleotide probes in an admixture. The probe library has a certain “complexity”, which is used herein to describe the number of distinct oligonucleotides in a probe library. For example, a library with a complexity of 4⁷, comprises 4⁷ (i.e., 16,384) distinct oligonucleotide probes. The term “complexity” does not describe the presence of more than one copy of each distinct oligonucleotide probes, but rather describes the number of unique probes in a library. The complexity of a library is determined by the number of random (e.g., degenerate) nucleotide combinations generated using a desired template probe sequence, wherein N, R, Q, S, W, X, Y, or Z (upper and lower case and with or without apostrophe neutral) is used to represent each of the nucleotides A, T/U, C, and G (note that the nucleotide(s) to be converted, w′, x′ y′ z′, can also be an A, T/U, C, or G). For example, if there are 2 random nucleotides, designated as n, n in a probe sequence and there are 4 possible DNA nucleotides (e.g., A, T, C, and G) for each n, the library has a complexity of 4², or 16 distinct oligonucleotides. Therefore, the library comprises all the possible combinations of A, T, C, and G (and optionally indiscriminate binding nucleotides, such as inosine (I)) for a set length of a probe in order for at least one probe to specifically hybridize with an unknown region on a target ssDNA molecule (i.e., knowledge of the target ssDNA sequence is not necessary for the methods described herein). The probe library(s) that are useful in the methods described herein exemplary probes are shown in FIG. 10. It should be noted that conversion can be performed starting from either the 3′ end or 5′ end of the target molecule. An exemplary probe for each type of conversion is described in the Detailed Description section. It should be understood that a skilled artisan can adapt the probe libraries for conversion to convert either the 5′ end or the 3′ end of a target molecule.

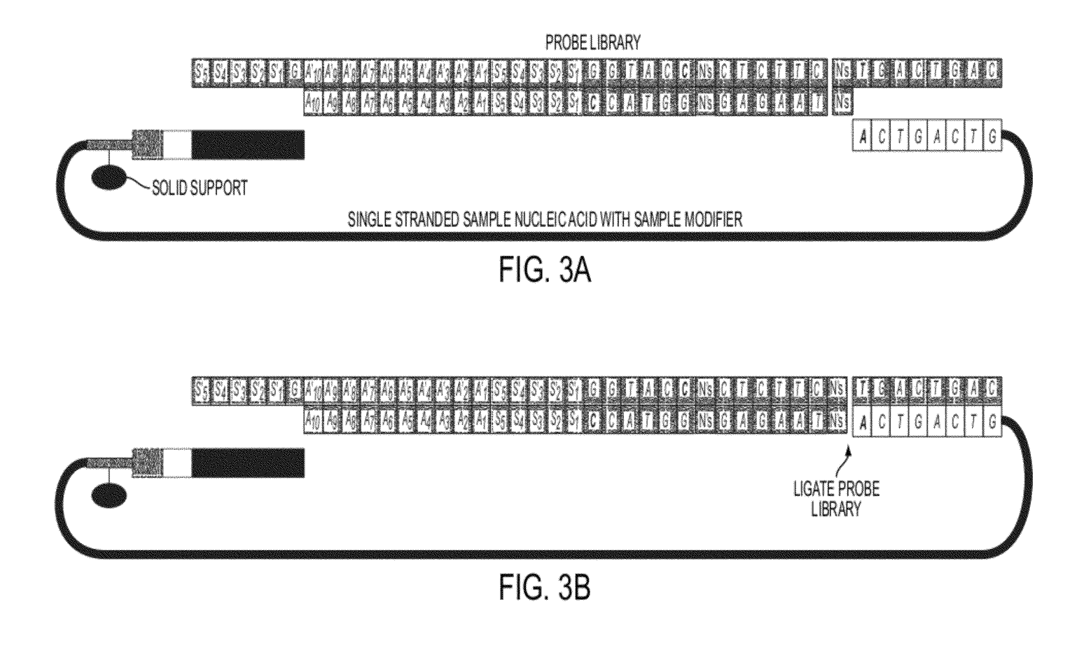

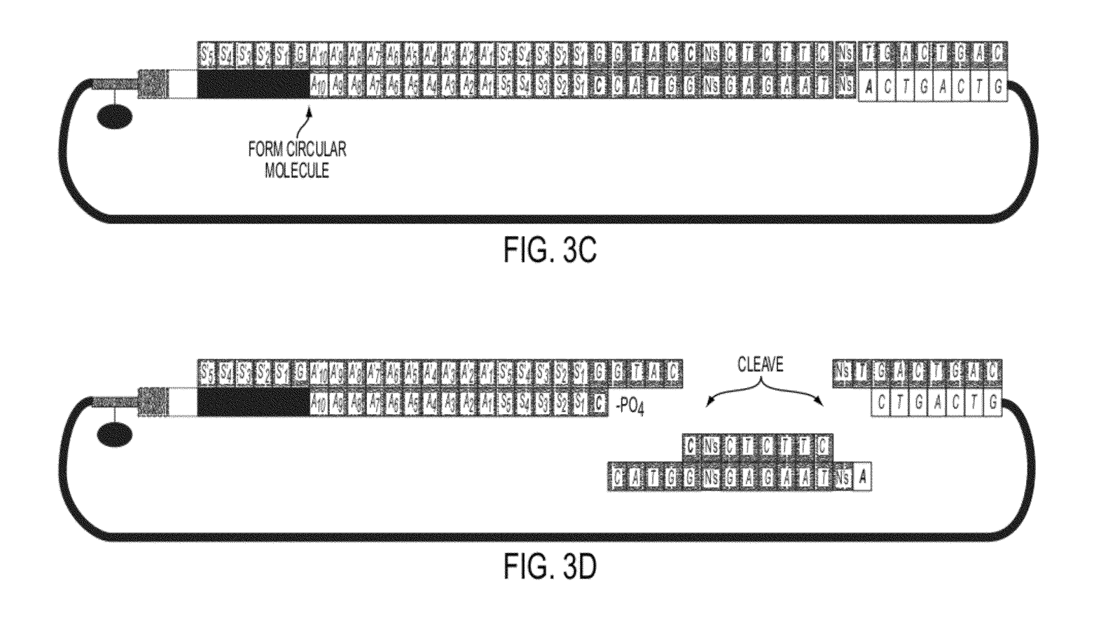

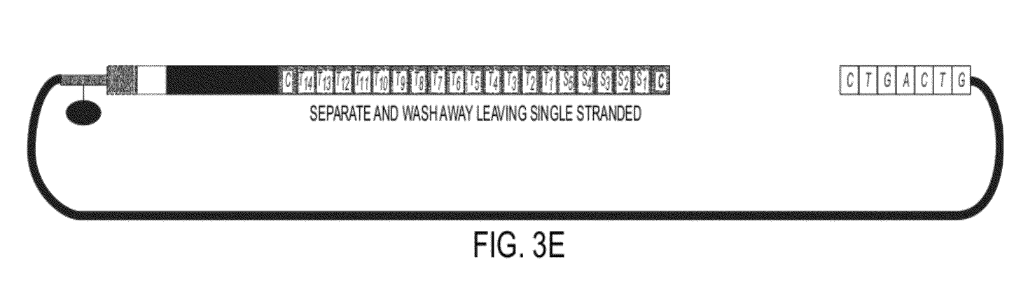

As used herein, the term “pre-specified nucleotide sequence” is used to describe a known nucleotide sequence that is ligated to one end of the target single-stranded nucleic acid to be converted, e.g., ssDNA, which is attached to one end (e.g., either the 5′ or the 3′ end) of a target ssDNA molecule (e.g., see FIGS. 3A-3E, wherein the pre-specified sequence designated as 5′-n′, s5′, s4′, s3′, s2′, s1′-3′ is attached to the 5′ end of the target ssDNA). The pre-specified nucleotide sequence is complementary to a nucleotide sequence incorporated into each probe and is used for the first round of sequence preserved DNA conversion.

The term “target ssDNA molecule” is used herein to describe a single-stranded DNA to be converted. The target ssDNA molecule can be derived from a double-stranded DNA molecule (e.g., a genomic DNA sample) that has been denatured from its native duplex conformation to a single-stranded conformation. The term “target ssDNA molecule” also encompasses fragments of a ssDNA molecule or short ssDNAs (e.g., 500 bp, 1 Kb, 2 Kb, 5 Kb, 16 Kb, etc.). It is also contemplated herein that a target single-stranded nucleic acid, e.g., RNA, can be converted with the methods disclosed herein. The term “target single-stranded nucleic acid” also encompasses single-stranded RNA. For illustration purposes, target ssDNA molecules are used throughout the description as an example of the methods described herein. One of skill in the art can readily adapt these methods for the conversion of RNA molecules, if desired.

As used herein, the term “specifically hybridize(s)” refers to the association between two single-stranded nucleic acid molecules of sufficiently complementary sequence to permit such hybridization under pre-determined conditions generally used in the art (sometimes termed “substantially complementary”). In one embodiment, one uses at least moderate stringency conditions. In another embodiment one uses high stringency conditions. In particular, the term refers to hybridization of an oligonucleotide with a substantially complementary sequence contained within a single-stranded DNA molecule of the invention, to the substantial exclusion of hybridization of the oligonucleotide with single-stranded DNA of non-complementary sequence.

As used herein, “complementary” refers to the broad concept of sequence complementarity between regions of two nucleic acid strands or between two regions of the same nucleic acid strand. For example, it is known that an adenine residue of a first nucleic acid region is capable of forming specific hydrogen bonds (“base pairing”) with a residue of a second nucleic acid region which is anti-parallel to the first region if the residue is thymine or uracil. Similarly, it is known that a cytosine residue of a first nucleic acid strand is capable of base pairing with a residue of a second nucleic acid strand, which is anti-parallel to the first strand, if the residue is guanine A first region of a nucleic acid is complementary to a second region of the same, or a different nucleic acid, if when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an anti-parallel fashion, at least about 50%, and at least about 75%, at least about 90%, or at least about 95%, or at least about 99% of the nucleotide residues of the first portion are capable of base pairing with nucleotide residues in the second portion. In one embodiment, all nucleotide residues (e.g., 100%) of the first portion are capable of base pairing with nucleotide residues in the second portion.

For ease of reference the strands of a duplex DNA molecule are denoted according to the position of the terminal phosphate group and the terminal hydroxyl group on the DNA strand. A DNA strand is referred to as a 5′-3′ directional strand and is denoted by a 5′ phosphate group and a 3′ hydroxyl group; this strand is depicted in the figures shown herein as the “upper” or “top” strand denoted by N, Q, R, S, W, X, Y, or Z. The complement to the 5′-3′ directional strand is denoted from left to right as a 3′-5′ directional strand and is depicted in the figures shown herein as the “lower” or “bottom” strand denoted by n′, q′, r′, s′, w′, x′, y′, or z′. Upper and lower case letters with or without apostrophe are shown for convenience only and add no specific content to the description. The 5′-phosphate may or may not be specifically depicted unless consideration of the presence or absence of the phosphate in a particular step is important.

As used herein, “stringent conditions” are conditions that permit specific hybridization of a substantially complementary oligonucleotide probe to a target ssDNA molecule to be converted, but does not permit non-complementary oligonucleotide probes to bind to a target ssDNA molecule. Stringency of hybridization and wash buffers can be altered by changing incubation temperatures or buffer compositions (e.g., salt concentrations, detergent, pH, etc.). Stringent hybridization conditions can vary (e.g., from salt concentrations of less than about 1M, more usually less than about 500 mM and in some embodiments less than about 200 mM) and hybridization temperatures can range (e.g., from as low as 0° C. to greater than 22° C., greater than about 30° C., and (most often) in excess of about 37° C.) depending upon the lengths and/or the nucleic acid composition of the oligonucleotide probes. Stringency may be increased, for example, by washing at higher temperatures (e.g., 55° C. or in some embodiments 60° C.) using an appropriately selected wash medium having an increase in sodium concentration (e.g., I×SSPE, 2×SSPE, 5×SSPE, etc.). If problems remain with cross hybridization, further increases in temperature can also be selected, for example, by washing at 65° C., 70° C., 75° C., or 80° C. Longer fragments may require higher hybridization temperatures for specific hybridization. The skilled artisan is aware of various parameters which may be altered during hybridization and washing, which will either maintain or change the stringency conditions (see e.g., Sambrook et al., 1989 “Molecular Cloning: a Laboratory Manual,” 2nd Edition, Cold Spring Harbor, N.Y., Cold Spring Harbor Laboratory Press, at 11.45). As several factors affect the stringency of hybridization, the combination of parameters is more important than the absolute measure of a single factor.

Described herein are inexpensive high throughput methods to convert a target single-stranded DNA (ssDNA) such that each nucleotide (or base) adenine (A), thymine (T), guanine (G) and cytosine (C) is converted to a pre-determined oligonucleotide code, with the sequential order preserved in the converted ssDNA. The conversion method is performed on a solid support to permit efficient sample processing throughout the conversion. One can also adapt this method to convert RNA by appropriate modification thereof. The target molecules may be labeled with a barcode to permit sample tracking. The method involves the use of an oligonucleotide probe library with repeated cycles of ligation and cleavage. At each cycle, one or more nucleotides on one end (e.g., either the 5′ end or the 3′ end) of a target, e.g., ssDNA, are ligated with the corresponding oligonucleotide code from the oligonucleotide probe library and the distal end of the probe library is ligated at the other end of the target ssDNA to form a circular DNA. The nucleotide(s) on the one end coded by the oligonucleotide library is then cleaved from that end exposing the next nucleotide(s) to be encoded in the subsequent cycles. Following the conversion method the converted molecule is hybridized to detectably labeled molecules forming a complex which enable the decoding of the converted DNA using optical detection while translocation through a nanopore. The detectably labeled molecules may be labeled with a bulky group which facilitates the stripping of the molecule from the converted molecule. The hybridized complex must be liberated from the solid support using methods which permit high yields and more importantly do not remove the hybridized detectably labeled molecules from the complex. The method does not require the use of DNA polymerases during the cycles, which eliminates the introduction of errors into the sequence via a polymerase (see e.g., T. Sjoblom et al., Science 314, 268 (2006)). One embodiment of the invention permits sequencing of e.g., an entire human genome in a relatively short time (e.g., no more than a couple of days, in some embodiments no more than a day.

In some embodiments, the method for conversion of a target nucleic acid comprises: (a) attaching a sample modifier comprising a moiety for immobilization to a solid support and a pre-specified sequence to a target nucleic acid, to provide a modified nucleic acid, (b) immobilizing the modified nucleic acid onto a solid support, (c) ligating a probe library comprising a pre-determined expanded code for one or more bases to the immobilized modified nucleic acid, (d) forming a circular molecule by circularizing the molecule produced in step (c), (e) cleaving the circular molecule with one or more restriction enzymes, (f) optionally separating and washing away the double-stranded portion leaving single-stranded nucleic acid on the solid support, and (g) repeating steps (c) to (f) two or more times wherein the conversion results in a converted molecule which can be used to determine the nucleotide sequence of the target nucleic acid.

In some embodiments, the converted nucleotides are converted into longer pre-determined oligonucleotide codes that can further bind to molecular beacons. The converted single-stranded nucleic acid molecule (e.g., ssDNA) can thus be sequenced, in one embodiment, through the use of a nanopore, wherein one bound molecular beacon is removed one at a time as the converted ssDNA strand moves through a nanopore. Removing a molecular beacon produces a flash of light, which translates to the sequence of a target single-stranded nucleic acid molecule. Since the longer pre-determined oligonucleotide codes (each code corresponding to each of the nucleotides A, C, T or G in e.g., a target ssDNA) are integrated into the target ssDNA molecule, the method described herein does not require detection at the single nucleotide level and thus overcomes one of the major challenges of nanopore-based sequencing. The conversion methods of the invention described herein permit rapid sequencing with any sequencing method useful at the single molecule level (i.e., sequencing is not limited to nanopore sequencing).

In some embodiments, a method for sequencing of a target nucleic acid comprises: (a) attaching a sample modifier comprising a moiety for binding to a solid support and a pre-specified sequence to a target nucleic acid to provide a modified nucleic acid, (b) immobilizing the modified nucleic acid onto a solid support, (c) ligating a probe library comprising a pre-determined expanded code for one or more bases to the immobilized modified nucleic acid, (d) forming a circular molecule by circularizing the molecule produced in step (c), (e) cleaving the circular molecule with one or more restriction enzymes, (f) optionally separating and washing away the double-stranded portion leaving single-stranded nucleic acid on the solid support, (g) repeating steps (c) to (f) two or more times, (h) hybridizing to detectably labeled molecules forming a complex, (i) detaching the complex from the solid support, and (j) translocating the complex through a nanopore, wherein the translocation produces detectable signals which can be used to determine the nucleotide sequence of the target nucleic acid.

In some embodiments, the optically detectable oligonucleotide probes comprising a fluorophore and a quencher which are not molecular beacons. In other embodiments, the optically detectable oligonucleotide probes comprise only a fluorophore. In some embodiments, optically detectable oligonucleotide are molecular beacons. In other embodiments, optically detectable oligonucleotide are not molecular beacons.

In some embodiments, removing an optically detectable oligonucleotide probe produces a flash of light, which translates to the sequence of a target single-stranded nucleic acid molecule. The flash of light produced is spectrally distinguishable into one of two colors and the each of the nucleotides A, C, T, G are defined using a binary code comprised of two color combinations, i.e., A color 1/color1, T color1/color 2, C color 2/color 1, and G color 2/color2.

One aspect of the methods described herein relates to DNA conversion however RNA may also be converted. The target nucleic acid (DNA/RNA) may first be fragmented to some desired length ranging from a few 100's to 1000's of bases. A sample modifier is attached to each strand permitting surface attachment and start site for the conversion process. Attaching the sample modifier can be achieved using a ligase enzyme.

One aspect of the conversion involves the formation of a circular molecule comprising a target single-stranded DNA (ssDNA) by ligating double-stranded or T-shaped probes to the target ssDNA, digesting with a Type IIS restriction enzyme, wherein digesting leads to the removal of at least one converted base from the target ssDNA while adding a longer oligonucleotide code representing the converted nucleotide(s). In addition, another aspect described herein relates to the use of an oligonucleotide probe library, comprising T-shaped probes, for the purpose of converting a ssDNA molecule.

Some embodiments of the methods disclosed herein relate to a method for converting a target single-stranded DNA (ssDNA) molecule starting at its 3′ end, such that the nucleotides adenine (A), guanine (G), cytosine (C), or thymine (T) of the target ssDNA molecule are converted to a predetermined oligonucleotide code and that the order of the nucleotides of the target ssDNA is preserved during conversion. The method involves multiple conversion cycles comprising the steps of: (a) contacting a target ssDNA having the pre-specified sequence 5′-n′, s5′, s4′, s3′, s2′, s1′-3′ at its 5′-end, wherein n′ can be A, C, G, or T and s1′, s2′, s3′, s4′, s5′ is the complementary sequence of S1, S2, S3, S4, S5 adjoining the predetermined oligonucleotide code (Xx′), with a probe library comprising a plurality of oligonucleotide probes, wherein each probe comprises a double-stranded DNA portion and a first and second single-stranded overhang, wherein the double-stranded DNA portion comprises a) the predetermined oligonucleotide code (Xx′) that uniquely corresponds to the nucleotide to be converted (x′) in the target ssDNA, b) a type IIS restriction enzyme recognition sequence, (R/r′) wherein a IIS enzyme can specifically bind to R/r′ and cleave outside of the recognition sequence to the 5′ side of the second single-stranded overhang of the probe and c) a second restriction enzyme recognition sequence (Q/q′) wherein a restriction enzyme binds to Q/q′ and cleaves within the recognition sequence. The first single-stranded overhang comprises the sequence 5′-S1, S2, S3, S4, S5-3′ that is complementary to the sequence the ssDNA (5′-s5′, s4′, s3′, s2′, s1′-3′). The second single-stranded overhang of the probe is represented by all four nucleotides in the probe library (x); wherein the second single-stranded overhang having the sequence 5′-X n, n, n, n-3′ comprises a nucleotide (X) that is complementary to the nucleotide to be converted (x′) followed by at least four positions that are represented by all four nucleotides in the probe library, and wherein contacting is performed under conditions that permit one of a plurality of probes in the library to bind and form a perfectly matched duplex with the target ssDNA molecule, (b) ligating both ends of the shorter strand of the bound probe in step (a) to the target ssDNA with a ligase, thereby forming a circular probe-target ssDNA complex, (c) contacting the ligated molecule of step (b) with a type IIS restriction enzyme that specifically recognizes the sequence R/r′ present in the double-stranded DNA portion of the probe in step (a), wherein the enzyme cleaves at least one nucleotide on the 3′ end of the target ssDNA to be converted, thereby removing the nucleotide from the 3′ end of the target ssDNA molecule; (d) contacting the cleaved molecule of (c) with a second restriction enzyme which specifically recognizes sequence Q/q′ and cleaves within sequence (Q/q′) liberating a short dsDNA fragment which is comprised of sequences from digested Q/q′, R/r′, and base x′ and (e) separating the double-stranded portion of the probe-target ssDNA complex, which was cleaved in step (c+d), and washing away the oligonucleotides from the unligated strand of the probe; wherein steps (a)-(e) yield a converted target ssDNA molecule comprising on its 5′ end 5′-n′, s5′, s4′, s3′, s2′, s1′, -3′, wherein n′ is one (or more) nucleotides from site Q/q′ resulting from the action of the cleavage by the second restriction enzyme.

Some embodiments of methods disclosed herein relate to a method for converting a target single-stranded DNA (ssDNA) molecule starting at its 3′ end, such that more than one nucleotide adenine (A), guanine (G), cytosine (C), or thymine (T) of the target ssDNA molecule at a time are converted to a predetermined oligonucleotide code and that the order of the nucleotides of the target ssDNA is preserved during conversion. The method involves multiple conversion cycles comprising the steps of: (a) contacting a target ssDNA having the pre-specified sequence 5′-n′, s5′, s4′, s3′, s2′, s1′-3′ at its 5′-end, wherein n′ can be A, C, G, or T and s1′, s2′, s3′, s4′, s5′ is the complementary sequence of S1, S2, S3, S4, S5 adjoining a predetermined oligonucleotide code (Ww′,Yy′, Xy′,Zz′), with a probe library comprising a plurality of oligonucleotide probes, wherein each probe comprises a double-stranded DNA portion and a first and second single-stranded overhang, wherein the double-stranded DNA portion comprises a) the predetermined oligonucleotide code (Ww′,Yy′,Xy′,Zz′) wherein Ww′, Yy′, Xx′, Zz′ are codes for the 4 bases and that uniquely corresponds to the order of the nucleotides to be converted (w′, x′, y′, z′) in the target ssDNA, b) a type IIS restriction enzyme recognition sequence, (R′/R) wherein a IIS enzyme can specifically bind to R′/R and cleave outside of the recognition sequence to the 5′ side of the second single-stranded overhang of the probe and c) a second restriction enzyme recognition sequence (Q/q′) wherein a restriction enzyme binds to Q/q′ and cleaves within the recognition sequence. The first single-stranded overhang comprises the sequence 5′-S1, S2, S3, S4, S5, that is the complementary sequence of the ssDNA (5′-s5′, s4′, s3′, s2′, s1′-3′). The second single-stranded overhang of the probe is represented by all four nucleotides in the probe library (w′, x′, y′, z′); wherein the second single-stranded overhang having the sequence 3′-w′, x′, y′, z′, n, n-5′ comprises a series of nucleotides of defined composition nucleotide (w, x, y, z) that is complementary to the nucleotides to be converted (w′, x′, y′, z′) followed by at least two positions that are represented by all four nucleotides in the probe library, and wherein contacting is performed under conditions that permit one of a plurality of probes in the library to bind and form a perfectly matched duplex with the target ssDNA molecule, (b) ligating both ends of the shorter strand of the bound probe in step (a) to the target ssDNA with a ligase, thereby forming a circular probe-target ssDNA complex, (c) contacting the ligated molecule of step (b) with a type IIS restriction enzyme that specifically recognizes the sequence R/r′ present in the double-stranded DNA portion of the probe in step (a), wherein the enzyme cleaves at after w′, x′, y′, z′ on the 3′ end of the target ssDNA to be converted, thereby removing the nucleotides w′, x′, y′, z′ from the 3′ end of the target ssDNA molecule; (d) contacting the cleaved molecule of (c) with a second restriction enzyme which specifically recognizes sequence Q/q′ and cleaves within sequence (Q/q′) liberating a short dsDNA fragment which is comprised of sequences from digested Q/q′, R/r′, and bases w′, x′, y′, z′ and (e) separating the double-stranded portion of the probe-target ssDNA complex, which was cleaved in step (c+d), and washing away the oligonucleotides from the unligated strand of the probe; wherein steps (a)-(e) yield a converted target ssDNA molecule comprising on its 5′ end 5′-n′, s5′, s4′, s3′, s2′, s1′, w′, x′, y′, z′-3′ wherein w′,x′,y′,z′ is the pre-determined oligonucleotide code corresponding to the converted nucleotides w′x′y′ z′ of the target ssDNA and wherein n′ is one (or more) nucleotides from site Q/q′ resulting from the action of the cleavage by the second restriction enzyme.

In some embodiments, upon completion of the conversion, the converted product is cleaved from the solid support by means other than disrupting the streptavidin:biotin interaction which affixes the ssDNA to the solid support using a labile linkage appended onto the 5′-end of the target ssDNA. The linkage maybe be cleaved using either enzymatic or chemical methods. The cleavage site is appended to the target ssDNA in a way that the cleavage position is located between the biotin moiety and the sequence 5′-n′, s5′, s4′, s3′, s2′, s1′-3′. Upon completion of the conversion process and hybridization of the detectable probes, the sample is treated with an enzyme or a chemical which acts upon the cleavage site to release the converted ssDNA from the solid support leaving the biotin:streptavidin interaction intact in preparation for analysis.

In some embodiments, one or more nucleotides can be converted at a time (e.g., one nucleotide x, which can be A, T, G, or C, can be converted, or multiple nucleotides representing any combination of A, T, C, or G can be converted (e.g., GA, or ATC, TGAC, etc.).

In some embodiments, each of the plurality of predetermined oligonucleotide codes on the double-stranded portion of the probe corresponds uniquely to the converted nucleotide (A, T, G, or C).

In some embodiments, each of the plurality of predetermined oligonucleotide codes on the double-stranded portion of the probe corresponds uniquely a single optically detectable oligonucleotide probe.

In some embodiments, each of the plurality of predetermined oligonucleotide codes on the double-stranded portion of the probe corresponds uniquely more than one optically detectable oligonucleotide probe.

In some embodiments, the oligonucleotide library comprises T-shaped probes.

In some embodiments, the optically detectable oligonucleotide probes are added before release from the support.

Some embodiments of the methods disclosed herein relate to a method for converting a target single-stranded DNA (ssDNA) molecule starting at its 5′ end, such that the nucleotides adenine (A), guanine (G), cytosine (C), or thymine (T) of the target ssDNA molecule are converted to a predetermined oligonucleotide code and that the order of the nucleotides of the target ssDNA is preserved during conversion. The method involves multiple conversion cycles comprising the steps of: (a) contacting a target ssDNA having the pre-specified sequence 3′-n′, s5′, s4′, s3′, s2′, s1′-5′ at its 3′-end, wherein n′ can be A, C, G, or T and s1′, s2′, s3′, s4′, s5′ is the complementary sequence of S1, S2, S3, S4, S5 adjoined to a predetermined oligonucleotide code (Xx′), with a probe library comprising a plurality of oligonucleotide probes, wherein each probe comprises a double-stranded DNA portion and a first and second single-stranded overhang, wherein the double-stranded DNA portion comprises a) the predetermined oligonucleotide code (Xx′) that uniquely corresponds to the nucleotide to be converted (x′) in the target ssDNA, b) a type IIS restriction enzyme recognition sequence, (R/r′) wherein a IIS enzyme can specifically bind to R/r′ and cleave outside of the recognition sequence to the 3′ side of the second single-stranded overhang of the probe and c) a second restriction enzyme recognition sequence (Q/q′) wherein a restriction enzyme binds to Q/q′ and cleaves within the recognition sequence. The first single-stranded overhang comprises the sequence 3′-S1, S2, S3, S4, S5-5′ that is complementary to the sequence of the ssDNA (3′-s5′, s4′, s3′, s2′, s1′-5′). The second single-stranded overhang of the probe is represented by all four nucleotides in the probe library (X); wherein the second single-stranded overhang having the sequence 3′-X, n, n, n, n-5′ comprises a nucleotide (X) that is complementary to the nucleotide to be converted (x′) followed by at least four positions that are represented by all four nucleotides in the probe library, and wherein contacting is performed under conditions that permit one of a plurality of probes in the library to bind and form a perfectly matched duplex with the target ssDNA molecule, (b) ligating both ends of the shorter strand of the bound probe in step (a) to the target ssDNA with a ligase, thereby forming a circular probe-target ssDNA complex, (c) contacting the ligated molecule of step (b) with a type IIS restriction enzyme that specifically recognizes the sequence R/r′ present in the double-stranded DNA portion of the probe in step (a), wherein the enzyme cleaves at least one nucleotide on the 5′ end of the target ssDNA to be converted, thereby removing the nucleotide from the 5′ end of the target ssDNA molecule; (d) contacting the cleaved molecule of (c) with a second restriction enzyme which specifically recognizes sequence Q/q′ and cleaves within sequence (Q/q′) liberating a short dsDNA fragment which is comprised of sequences from digested Q/q′, R/r′, and base x′ and (e) separating the double-stranded portion of the probe-target ssDNA complex, which was cleaved in step (c+d), and washing away the oligonucleotides from the unligated strand of the probe; wherein steps (a)-(e) yield a converted target ssDNA molecule comprising on its 3′ end 3′-n′, s5′, s4′, s3′, s2′, s1′, x′-5′ wherein x′ is the pre-determined oligonucleotide code corresponding to the converted nucleotide x′ of the target ssDNA and wherein n′ is one (or more) nucleotides from site Q/q′ resulting from the action of the cleavage by the second restriction enzyme.

In some embodiments disclosed herein, upon completion of the conversion, the converted product is cleaved from the solid support by means other than disrupting the streptavidin:biotin interaction which affixes the ssDNA to the solid support using a labile linkage appended onto the 3′-end of the target ssDNA. The linkage maybe be cleaved using either enzymatic or chemical methods. The cleavage site is appended to the target ssDNA in a way that the cleavage position is located between the biotin moiety and the sequence 3′-n, s5′, s4′, s3′, s2′, s1′-5′.

Some embodiments disclosed herein relate to a method for converting a target single-stranded DNA (ssDNA) molecule starting at its 5′ end, such that more than one nucleotide adenine (A), guanine (G), cytosine (C), or thymine (T) of the target ssDNA molecule at a time are converted to a predetermined oligonucleotide code and that the order of the nucleotides of the target ssDNA is preserved during conversion. Upon completion of the conversion, the converted product is cleaved from the solid support by means other than disrupting the streptavidin/biotin interaction which affixes the ssDNA to the solid support using a labile linkage appended onto the 3′-end of the target ssDNA. The linkage maybe be cleaved using either enzymatic or chemical methods. The cleavage site is appended to the target ssDNA in a way that the cleavage position is located between the biotin moiety and the sequence 3′-n′, s5′, s4′, s3′, s2′, s1′-5′. The method involves multiple conversion cycles comprising the steps of: (a) contacting a target ssDNA having the pre-specified sequence 3′-n′, s5′, s4′, s3′, s2′, s1′-5′ at its 3′-end, wherein n′ can be A, C, G, or T and s1′, s2′, s3′, s4′, s5′ is the complementary sequence of S1, S2, S3, S4, S5 adjoined to a predetermined oligonucleotide code (Ww′, Yy′, Xx′,Zz′), with a probe library comprising a plurality of oligonucleotide probes, wherein each probe comprises a double-stranded DNA portion and a first and second single-stranded overhang, wherein the double-stranded DNA portion comprises a) the predetermined oligonucleotide code (Ww′, Yy′,Xx′,Zz′) wherein Ww′, Yy′, Xx′, Zz′ are codes for the 4 bases and that uniquely corresponds to the order of the nucleotides to be converted (w′, x′, y′, z′) in the target ssDNA, b) a type IIS restriction enzyme recognition sequence (R/r′) wherein a IIS enzyme can specifically bind to R/r′ and cleave outside of the recognition sequence to the 3′ side of the second single-stranded overhang of the probe and c) a second restriction enzyme recognition sequence (Q/q′) wherein a restriction enzyme binds to Q/q′ and cleaves within the recognition sequence. The first single-stranded overhang comprises the sequence 3′-S1, S2, S3, S4, S5-5′, that is complementary to the sequence of the ssDNA (3′-S5′, S4′, S3′, S2′, S1′-5). The second single-stranded overhang of the probe is represented by all four nucleotides in the probe library (w′, x′, y′, z′); wherein the second single-stranded overhang having the sequence 5′-w′, x, y′, z′,n, n-3′ comprises a series of nucleotides of defined composition nucleotide (w, x, y, z) that is complementary to the nucleotides to be converted (w′, x′, y′, z′) followed by at least two positions that are represented by all four nucleotides in the probe library, and wherein contacting is performed under conditions that permit one of a plurality of probes in the library to bind and form a perfectly matched duplex with the target ssDNA molecule, (b) ligating both ends of the shorter strand of the bound probe in step (a) to the target ssDNA with a ligase, thereby forming a circular probe-target ssDNA complex, (c) contacting the ligated molecule of step (b) with a type IIS restriction enzyme that specifically recognizes the sequence R/r′ present in the double-stranded DNA portion of the probe in step (a), wherein the enzyme cleaves at after w′, x′, y′, z′ on the 5′ end of the target ssDNA to be converted, thereby removing the nucleotides w′, x′, y′, z′ from the 5′ end of the target ssDNA molecule; (d) contacting the cleaved molecule of (c) with a second restriction enzyme which specifically recognizes sequence Q/q′ and cleaves within sequence (Q/q′) liberating a short dsDNA fragment which is comprised of sequences from digested Q/q′, R/r′, and bases w′, x′, y′, z′ and (e) separating the double-stranded portion of the probe-target ssDNA complex, which was cleaved in step (c+d), and washing away the oligonucleotides from the unligated strand of the probe; wherein steps (a)-(e) yield a converted target ssDNA molecule comprising on its 3′ end 3′-S′5, S′4, S′3, S′2, S′1, w, x, y, z′-5′, wherein w′,x′,y′,z′ is the pre-determined oligonucleotide code corresponding to the converted nucleotides w′x′y′z′ of the target ssDNA. Upon completion of the conversion process the sample is treated with an enzyme or a chemical to release the converted ssDNA from the solid support in preparation for analysis.

In some embodiments, steps (a)-(e) are repeated more than once, for example, the number of cycles may be 10, 50, 100, 200, 500, 1000 or more.

In some embodiments, the target ssDNA molecule is immobilized on a solid support or by any other means to ensure that the target ssDNA is not washed away in step (e) as described above.

In some embodiments, the target ssDNA is immobilized on a surface using streptavidin:biotin.

In some embodiments, the sample modifier containing the pre-specified sequence on the target ssDNA molecule further comprises a labile moiety different than by which the ssDNA target is immobilized on the surface and which when chemically treated is cleaved and detaches the converted molecule from the surface. The chemical treatment may involve the use of enzymes or chemicals.

In some embodiments, the cleavage site is comprised of a site for an enzyme action, for example, a restriction enzyme which binds to the converted molecule and cleaves between the site of the surface attachment group and the conversion product. The restriction enzyme is in some embodiments different than either restriction enzyme used in the conversion process. Included in this embodiment of cleavage is the use of a specific oligonucleotide which hybridizes to the converted molecule forming a dsDNA in the region of the restriction enzyme recognition sequence.

In some embodiments, the cleavage site is comprised of natural nucleotides or nucleotide analogs which are the target for an enzyme, for example, uracil-N-glycosylase which cleaves nucleic acid at the site of deoxyuracil bases inserted into the base sequence at the targeted cleavage site of the converted molecule and cleaves between the site of the surface attachment group and the conversion product.

In some embodiments, the labile moiety is comprised of one or more ribonucleotides might be inserted which are cleaved by either the action of an enzyme, e.g., RNase, or chemically, e.g., by using a chemical such as periodate.

In some embodiments, the labile moiety is comprised of a site of chemical action, for example, the labile moiety is comprised of a thiophosphonate linkage which is cleaved by action of silver ions.

In some embodiments, the labile moiety is comprised of more than one labile cleavage site, only a single site has to undergo cleavage to effect release of the converted molecule from the solid support.

In some embodiments, the pre-specified sequence on the target ssDNA molecule further comprises a recognition site for a restriction enzyme which results in the release of the converted molecule from the support.

In some embodiments, the sample modifier on the target ssDNA molecule further comprises a barcode which identifies target ssDNA from specific samples and permits the mixing of multiple samples prior to conversion. Following conversion and reading of the sequences, those sequencing arising from samples with the same barcode are compiled into a single sequence for each sample.

In some embodiments, the barcode is comprised of 4-10 individual nucleotide bases the order of which identifies a specific sample. Barcodes in this configuration must be converted by the conversion process into the expanded pre-determined expanded codes for each base.

In some embodiments, the barcode is preceded, followed by, or both by a series of more than one bases which identify the starting and stopping sequence of the barcode. These bases are also converted into their corresponding pre-determined expanded code.

In some embodiments, the barcode may be comprised of 4-10 pre-determined expanded codes used in the conversion process. Barcodes in this configuration are not required to be further converted via the conversion process and may be read directly following hybridization of the labeled probes and sequencing on a nanopore.

In some embodiments, the pre-determine expanded codes for the barcode may be preceded, followed by, or both a series of more than one expanded pre-determine codes which identify the starting and stopping sequence of the barcode.

In some embodiments, the sample modifier on the target ssDNA molecule further comprises a tag sequence which identifies the end, i.e. the 5′-end or the 3′-end, of the converted ssDNA to permit orientation of the sequence with respect to the target ssDNA when aligned.

In some embodiments, the tag sequence is comprised of either a sequence of individual bases which are converted or a series of pre-determined expanded codes.

In some embodiments, the tag sequence is the barcode.

In some embodiments, the type IIS restriction enzyme site is selected from, but not limited to, the group consisting of: Alwl, BccI, BsmAl, Earl, Mlyl, Plel, Bmrl, Bsal, BsmBl, BtsCI, Faul, HpyAV, MnII, Sapl, BbSl, BciVI, Hphl, MboII, Bfual, BspMI, SfaNI, Hgal, Bbvl, EarI, Ecil, Fokl, BceAI, BsmFI, BtgZI, Bpml, BpuEI, BsgI, AclWI, Alw261, Bst61, BstMAI, Eaml1041, Ksp6321, PpS1, SchI, Bfil, Bso31I, BspTNI, Eco31I, Esp31, Faul, Smul, Bful, Bpil, BpuAI, BstV21, AsuHPI, Acc361, LweI, Aarl, BseMII, TspDTI, TspGWI, BseXI, BstVlI, Eco571, Eco57MI, Gsul, Psrl, and Mmel site.

In some embodiments, the recognition sequences for the restriction enzyme and the Type IIS maybe be spaced apart, for example, the spacer can be from 1-10 nucleotides.

In some embodiments, the restriction enzymes which cleave at sequences Q/q′ and R/r′ are added sequentially in a two-step process.

In some embodiments, the restriction enzymes which cleave at sequences Q/q′ and R/r′ are combined in a single-step process.

In some embodiments, the codes, for example, Ww′,Xx′, Yy′, Zz′ range from approximately 4 nucleotides to approximately 30 nucleotides each in length.

In some embodiments, Ww′,Xx′, Yy′, Zz′ are each 12-16 nucleotides in length.

In some embodiments, the codes, for example, Ww′,Xx′, Yy′, Zz′ are molecular beacons.

In some embodiments, the codes, for example, Ww′,Xx′, Yy′, Zz′ are not molecular beacons.

In some embodiments, the codes, for example, Ww′,Xx′, Yy′, Zz′ are comprised of binding sites for more than one optically detectable oligonucleotide probe.

In some embodiments, the codes, for example, Ww′,Xx′, Yy′, Zz′ are comprised of two binding sites for two color optically detectable oligonucleotide probes.

In some embodiments, the length of each overhang is determined by the length necessary to form a specific duplex between an overhang of the probe and one end of the target ssDNA, i.e. the overhang can be of any length.

In some embodiments, the first overhang ranges from approximately 3 nucleotides to approximately 12 nucleotides in length, or any range in between, e.g., 4 nucleotides to 12 nucleotides, 4 to 11 nucleotides, or 5 to 12 nucleotides, or 5 to 11 nucleotides, or 5 to 10 nucleotides in length etc.

In some embodiments, the second overhang ranges from approximately 3 nucleotides to approximately 12 nucleotides in length. or any range in between, e.g., 4 nucleotides to 12 nucleotides, 4 to 11 nucleotides, or 5 to 12 nucleotides, or 5 to 11 nucleotides, or 5 to 10 nucleotides in length etc.

In some embodiments, the target ssDNA ranges from approximately 50 nucleotides to approximately 3,000,000 nucleotides in length.

In some embodiments, a plurality of target ssDNA molecules are converted at the same time.

In some embodiments, the conversion is performed on a sample comprising a heterogeneous mixture of target ssDNA nucleic acids.

In some embodiments, a polymerase enzyme is not used at any step (a)-(e) in the method.

In some embodiments, the probe library has a complexity ranging from 16 to 1,048,576 distinct oligonucleotides.

In some embodiments, the target ssDNA molecule is derived from a mammal.

In some embodiments, the mammal is a human.

In some embodiments, the converted ssDNA molecule is sequenced at the single molecule level.

In some embodiments, sequencing comprises the use of one or more labeled molecular beacons.

In some embodiments, the labeled molecular beacon is a fluorescent molecular beacon.

In some embodiments, the fluorescent molecular beacon binds to a code sequence (e.g., Ww′,Xx′, Yy′, Zz′) of the converted ssDNA molecule.

In some embodiments, the fluorescent molecular beacons are labeled with 4 optically resolvable fluorophores, each fluorophore encoding for a different base A, C, G, or T.

In some embodiments, the fluorescent detectably labeled molecules are not molecular beacons and are labeled with 4 optically resolvable fluorophores, each fluorophore encoding for a different base A, C, G, or T.

In some embodiments, the fluorescent detectably labeled molecules comprise a bulky moiety different from either the fluorophore or quencher.

In some embodiments, the bulky group is any molecule which has an average size wherein the diameter is greater than the average opening in a nanopore array.

In some embodiments, the bulky group is any molecule which has an average size wherein the diameter is at least 1.2 times greater than the average opening in a nanopore array.

In some embodiments, the bulky group is any molecule which has an average size wherein the diameter is at least 2 times greater than the average opening in a nanopore array.

In some embodiments, the bulky group is any molecule which has an average size wherein the diameter is at least 5 times greater than the average opening in a nanopore array.

In some embodiments, the fluorescent detectably labeled molecules comprise a bulky moiety which may be either the fluorophore or quencher, e.g., a quantum dot.

In some embodiments, the fluorescent detectably labeled molecules comprise a bulky moiety different from either the fluorophore or quencher which may be a protein.

In some embodiments, the Xx′ (e.g., Ww′, Xx′, Yy′, Zz′) sequence of the converted ssDNA molecule having a bound optically detectable oligonucleotide probe(s) is directed through a nanopore of diameter <2 nm, wherein the bound optically detectable oligonucleotide probe(s) is removed as the converted ssDNA molecule passes through the nanopore, wherein removal of the optically detectable oligonucleotide probe(s) produces a flash of light, wherein the order of light flashes yields the sequence of the target ssDNA sequence.

In some embodiments, the bulky group is directed through a nanopore of diameter <10 nm, wherein the bound optically detectable oligonucleotide probe(s) is removed as the converted ssDNA molecule passes through the nanopore.

Another aspect described herein is an oligonucleotide probe library comprising T shaped probes useful for the methods of DNA conversion described herein, e.g., the T-shape being defined by an upper and lower oligonucleotide of different lengths and the longer strand having both ends protruding over the shorter strand.

Another aspect described herein is a method for converting a target single-stranded DNA (ssDNA) molecule starting at its 3′ end such that the nucleotides adenine (A), guanine (G), cytosine (C), or thymine (T) of the target ssDNA molecule are converted to a predetermined oligonucleotide code, and that the order of the nucleotides of the target ssDNA is preserved during conversion. The method comprises the steps of: (a) contacting a target ssDNA molecule having a pre-specified nucleotide sequence on its 5′ end, S1, S2, S3, S4, S5 with a probe library and competitive blocking molecule, wherein contacting is performed under conditions that permit only one end of a probe in the probe library to hybridize to the 3′ end of the target ssDNA, and one portion of the competitive blocking molecule to hybridize to the 5′ overhang of the probe library molecule comprising the predetermined sequence, S1, S2, S3, S4, S5; (b) ligating the hybridized probes of step (a) such that only probes on the 5′ end are ligated to the 3′ end of the target ssDNA sequence; (c) exposing the ligated molecule of step (b) to a high temperature, thereby separating the blocking molecule from the ligated probe of the probe library with the ssDNA target; (d) hybridizing and ligating the 3′ end of the ligated probe from the probe library to the 5′ end of ssDNA comprising the predetermined sequenced, S1, S2, S3, S4, S5, thereby forming a circular molecule. (e) contacting the ligated molecule of step (d) with a type IIS restriction enzyme, wherein the type IIS restriction enzyme cleaves after at least one nucleotide on the 3′ end of the target ssDNA to be converted thereby removing the nucleotide to be converted from the 3′ end of the target ssDNA molecule; (f) contacting the molecule of step (e) with a second restriction enzyme which results in generation of the predetermined sequence on the 5′ end of the ssDNA, S1, S2, S3, S4, S5; and (g) separating the double-stranded portion of each of the ligated and cut probes of site (e+f) from the target ssDNA and washing away the unligated strands of the probes; wherein steps (a)-(g) yield a converted target ssDNA molecule comprising, on its 5′ end, the predetermined oligonucleotide code from the probe library corresponding to the converted nucleotide(s) of the target ssDNA.

Another aspect described herein is a method for converting a target single-stranded DNA (ssDNA) molecule starting at its 5′ end with a probe library and competitive blocking molecule.

In some embodiments, the use of a competitive blocking molecule is described which competes for binding at the first single-stranded overhang comprising the sequence 3′-S1, S2, S3, S4, S5-5′, that is complementary to the sequence of the ssDNA (3′-s5′, s4′, s3′, s2′, s1′-5′).

In some embodiments, a competitive blocking molecule is used which competes for binding at the first single-stranded overhang comprising the sequence 5′-S1, S2, S3, S4, 55-3′, that is complementary to the sequence of the ssDNA (5′-S5′, S4′, S3′, S2′, S1′-3′).

In some embodiments, the competitive blocking molecule is an oligonucleotide comprising DNA monomers (i.e., DNA nucleotides), an oligonucleotide comprising RNA monomers (i.e., RNA nucleotides), an oligonucleotide comprising PNA monomers, an oligonucleotide comprising LNA monomers, or mixtures and analogs thereof.

Described herein is a method for sequentially converting each nucleotide of a target single-stranded nucleic acid, such as DNA or RNA, to a pre-determined code, the product of such method accurately representing the order of nucleotides adenine (A), thymine (T)/uracil (U), guanine (G) and cytosine (C), of a target single-stranded nucleic acid sequence. Following conversion, each nucleotide of the target sequence (e.g., a target ssDNA) is represented by a pre-determined oligonucleotide code sequence. The target nucleic acid following conversion has been converted into a synthetic representation (i.e., a polymer of pre-determined oligonucleotide code sequences representing the order of bases in the target nucleic acid sequence) that can further bind optically detectable labeled molecules. One aspect of the methods described herein relates to DNA conversion that requires the formation of a circular molecule and leads to the removal of the converted base(s) from one end of the ssDNA. In addition, another aspect described herein relates to the use of an oligonucleotide probe library, comprising T-shaped probes, for the purpose of converting a ssDNA molecule.

In some embodiments, a method for conversion of a target nucleic acid involves the process of: (a) attaching a sample modifier comprising a moiety for immobilization to a solid support and a pre-specified sequence to a target nucleic acid to provide a modified nucleic acid, (b) immobilizing the modified nucleic acid onto a solid support, (c) ligating a probe library comprising a pre-determined expanded code for one or more bases to the immobilized modified nucleic acid, (d) forming a circular molecule by circularizing the molecule produced in step (c), (e) cleaving the circular molecule with one or more restriction enzymes, (f) optionally separating and washing away the double-stranded portion leaving single-stranded nucleic acid on the solid support, (g) repeating steps (c) to (f) two or more times, wherein the conversion results in a converted molecule which can be used to determine the nucleotide sequence of the target nucleic acid.

In some embodiments such conversion permits the converted single-stranded molecule to be sequenced through the use of nanopore sequencing. In this embodiment, one bound optically detectable molecule is removed one at a time in sequential order as the converted synthetic representation moves through a nanopore. Removing an optically detectable molecule produces a flash of light, which represents the order of the predetermined code, and also translates to the order of the nucleotides in the target ssDNA. This system has several advantages: (a) the sequence of the target ssDNA can be unknown, (b) no polymerase or amplification step is necessary, (c) a gel separation system is not required for the practice of the methods described herein, and (d) the system can be automated for rapid sequencing. The method of conversion of a target ssDNA described herein permits rapid sequencing at the single molecule level. In one embodiment, a target ssDNA can be sequenced in less than one week; in some embodiments the target ssDNA molecule is sequenced in less than 72 hours, less than 48 hours, less than 24 hours, less than 12 h, less than 6 hours, less than 2 hours or even less than one hour (e.g., 45 minutes, 30 minutes, 15 minutes, etc.).

In some embodiments, a method for sequencing of a target nucleic acid comprises the steps of: (a) attaching a sample modifier comprising a moiety for binding to a solid support and a pre-specified sequence to a target nucleic acid to provide a modified nucleic acid, (b) immobilizing the modified nucleic acid onto a solid support, (c) ligating a probe library comprising a pre-determined expanded code for one or more bases to the immobilized modified nucleic acid, (d) forming a circular molecule by circularizing the molecule produced in step (c), (e) cleaving the circular molecule with one or more restriction enzymes, (f) optionally separating and washing away the double-stranded portion leaving single-stranded nucleic acid on the solid support, (g) repeating steps (c) to (f) two or more times, (h) hybridizing the converted molecule to detectably labeled molecules forming a complex, (i) detaching the complex from the solid support, and (j) translocating the complex through a nanopore, wherein the translocation produces detectable signals which can be used to determine the nucleotide sequence of the target nucleic acid.

### Target Nucleic Acid Templates

### Sources

The methods described herein are contemplated for the conversion of any single-stranded nucleic acid molecule including, for example RNA and ssDNA. Target single-stranded nucleic acids can be derived from a variety of sources including, for example genomic DNA, double-stranded DNA, cDNA, mRNA, tRNA, rRNA, siRNA, miRNA, shRNA or reverse transcribed DNA. The single-stranded DNA can be prepared from a double-stranded DNA that occurs naturally e.g., genomic DNA, or alternatively can be engineered, for example a cDNA construct. It is not necessary that the target nucleic acid molecule contain a region of known sequence, as the methods described herein permit sequencing of a completely unknown sequence. In addition, the target nucleic acid need not be an entire genomic sequence or full-length RNA molecule, but rather a target nucleic acid can be a shorter sequence (i.e., 500 bp, 1 Kb, 2 Kb, 16 Kb). However, conversion of an entire genome is also contemplated herein, as well as fragmented genomic DNA. The methods of conversion described herein can be used, for example to convert an entire genome that has been fragmented into smaller pieces, such that the initial DNA sequence can be reconstructed from the various fragment sequences.

Target nucleic acid molecules can be isolated from any species by methods known to those skilled in the art. Target nucleic acids include but are not limited to those comprised by bacteria, viruses, fungi, plants, animals, etc., including humans.

Nucleic acid samples can be derived from a biological sample. Some non-limiting examples of biological samples include a blood sample, a urine sample, a semen sample, a lymphatic fluid sample, a cerebrospinal fluid sample, a plasma sample, a serum sample, a pus sample, an amniotic fluid sample, a bodily fluid sample, a stool sample, a biopsy sample, a needle aspiration biopsy sample, a swab sample, a mouthwash sample, a cancer sample, a tumor sample, a tissue sample, a cell sample, a cell lysate sample, a crude cell lysate sample, a forensic sample, an environmental sample, an archeological sample, an infection sample, a nosocomial infection sample, a community-acquired infection sample, a biological threat sample, a production sample, a drug preparation sample, a biological molecule production sample, a protein preparation sample, a lipid preparation sample, a carbohydrate preparation sample, or any combination thereof. Other non-limiting examples of biological samples include a bacterial colony, a bacterial cell, a bacteriophage plaque, a bacteriophage, a virus plaque, a virus, a yeast colony, a yeast cell, a baculovirus plaque, a baculovirus, a biological agent, an infectious biological agent, a eukaryotic cell culture, a eukaryotic cell, a culture of transiently transfected eukaryotic cells, or a transiently transfected eukaryotic cell.

In one embodiment the target DNA molecule is derived from an individual in need of rapid sequencing analysis, for example an individual to be pre-screened for genetic polymorphisms prior to being prescribed a drug by a clinician.

In one embodiment the target DNA molecule is derived from an infected individual, for example one HIV positive individual considered for an antiviral therapy, for which a large number of HIV genomes need to be sequenced.

Preparation of Target ssDNA

In one embodiment the nucleic acid to be converted is a DNA molecule. Single-stranded DNA molecules can be prepared for conversion in a variety of ways. In cases when a target DNA is obtained in a double-stranded form (e.g., from a biological sample), the DNA can be fragmented into smaller pieces and denatured to yield single-stranded fragments. For example, by treating a double-stranded DNA (dsDNA) with DNase, sonication, vortexing, or other similar techniques nucleic acid molecules can be fragmented into pieces. Denaturation can be performed, for example by heating a target dsDNA to approximately 95° C. Such techniques are known to those of skill in the art. By adjusting the parameters of these techniques, it is possible to adjust the average size of the target DNA fragments. These methods are relatively non-specific with respect to where they cut/break the DNA molecule so that generally DNA pieces are obtained that are cut/broken throughout the entire sequence.

A pre-specified sequence is advantageous for the conversion methods described herein and in one embodiment can be attached to either end of a target ssDNA molecule (FIG. 1). The attachment may be performed before denaturing the target dsDNA to ssDNA using for example T4 DNA ligase (NEB, Ipswich, Mass.) or after denaturing using for example T4 RNA ligase (NEB, Ipswich, Mass.). Various ligases are available and methods for ligation are well known to one of skill in the art.

In an alternate embodiment, the genome is sheared by mechanical means or enzyme cleavage to produce fragmented dsDNA. Some restriction enzymes such as EcoRV (NEB, Ipswich, Mass.) cleave to produce blunt ends. Alternatively, the ends of the dsDNA molecule are converted to blunt ends with enzymes such as E. coli DNA polymerase I large fragment (Klenow fragment) or T4 DNA polymerase. A phosphatase may be removed to prevent self-ligation of the dsDNA. A sample modifier comprising a pre-specified oligonucleotide sequence, a surface attachment moiety (e.g., a biotin moiety such as biotin) and a 5′-phosphate, is then ligated to the target dsDNA fragments using a T4 DNA ligase. The DNA is then treated (e.g., by heating) to separate the two strands and produce single-stranded DNA fragments wherein the strand comprising the biotin will be subjected to the conversion process. Alternatively, the biotin labeled dsDNA is exposed to a support consisting of streptavidin, then exposed to elevated temperatures producing ssDNA from dsDNA and the surface retaining only ssDNA with biotin. The strand comprising the biotin remaining on the support will be subjected to the conversion process. Methods for these steps are well known to one of skill in the art.

In an alternate embodiment, a sample modifier comprising a pre-specified oligonucleotide tag, a surface attachment moiety (e.g., a biotin moiety such as biotin) and a labile cleavable linkage is then ligated to the target dsDNA fragments using a T4 DNA ligase (FIG. 1B).

The oligonucleotide comprising the sample modifier may also be comprised of one or more sequences which have functional utility in the conversion process or following conversion. In a preferred configuration, the sample modifier oligonucleotide comprises: (i) the pre-determined sequence, (ii) a moiety for surface attachment, for example a biotin which attaches to streptavidin/avidin/neutravidin coated surfaces, which aides in the conversion process of performing stepwise chemical reactions and washing, (iii) a barcode which permits tracking specific ssDNA back to sample of origin and permits mixing of samples prior to the conversion process, and (iv) a chemically labile site(s) which permit the converted ssDNA to be released from the support in preparation for sequencing using a nanopore (FIG. 1B Preferred multi-sample composition).

### Solid Supports

In one embodiment of the present invention, a target nucleic acid is immobilized to a solid substrate or support. The immobilization of a target single-stranded nucleic acid permits both removal of unincorporated probes and separate enzyme treatments to be performed with intervening wash steps without substantial loss of target single-stranded nucleic acid fragments during the process of conversion. The immobilization has the additional advantage of facilitating spatial separation of individual target ssDNA molecules so that during the conversion process circular intermediate product is produced as opposed to linear concatemers or polymers.

In its simplest version, the solid support comprises a plastic tube or microwell plate coated with streptavidin to which biotinylated target ssDNA sequences bind. In one embodiment of the invention, the target ssDNA is anchored to a streptavidin coated solid support, such as a magnetic particle, polymeric microsphere, filter material, or the like, which permits the sequential application of reagents without complicated and time-consuming purification steps.

A variety of other solid substrates can be used, including, without limitation, the following: cellulose; nitrocellulose; nylon membranes; controlled-pore glass beads; acrylamide gels; polystyrene matrices; activated dextran; avidin/streptavidin-coated polystyrene beads; agarose; polyethylene; functionalized plastic, glass, silicon, aluminum, steel, iron, copper, nickel, and gold; tubes; wells; micro titer plates or wells; slides; discs; columns; beads; membranes; well strips; films; chips; and composites thereof. In one embodiment, a portion of the surface of a solid substrate is coated with a chemical functional group to allow for covalent binding of, for example the target ssDNA, to the surface of the solid substrate. Solid substrates with the functional group already included on the surface are commercially available. In addition, the functional groups may be added to the solid substrates by the practitioner.

A number of methods can be used to couple e.g., a target ssDNA to a solid substrate, including, without limitation: covalent chemical attachment; biotin:avidin/streptavidin/neutravidin; and UV irradiation (see for example, Conner et al., Proc. Natl. Acad. Sci. 80 (1):278-282 (1983); Lockley et al., Nucleic Acids Res. 25 (6):1313-1314 (1997), which are hereby incorporated by reference in their entirety).

A target ssDNA solid substrate linkage can include, without limitation, the following linkage types: disulfide; carbamate; hydrazone; ester; (N)-functionalized thiourea; functionalized maleimide; streptavidin or avidin/biotin; mercuric-sulfide; gold-sulfide; amide; thiolester; azo; ether; and amino.

If a solid substrate is made of a polymer, it can be produced from, without limitation, any of the following monomers: acrylic acid; methacrylic acid; vinylacetic acid; 4-vinylbenzoic acid; itaconic acid; allyl amine; allylethylamine; 4-aminostyrene; 2-aminoethyl methacrylate; acryloyl chloride; methacryloyl chloride; chi oro styrene; dischlorostyrene; 4-hydroxystyrene; hydroxymethyl styrene; vinylbenzyl alcohol; allyl alcohol; 2-hydroxyethylmethacrylate; poly(ethylene glycol) methacrylate; and mixtures thereof, together with one of the following monomers: acrylic acid; acrylamide; methacrylic acid; vinylacetic acid; 4-vinylbenzoic acid, itaconic acid; allyl amine; allylethylamine; 4-aminostyrene; 2-aminoethyl methacrylate; acryloyl chloride; methacryloyl chloride; chi oro styrene; dichlorostyrene; 4-hydroxystyrene; hydroxymethyl styrene; vinylbenzyl alcohol; allyl alcohol; 2-hydroxyethylmethacrylate; poly(ethylene glycol) methacrylate; methyl acrylate; methyl methacrylate; ethyl acrylate; ethyl methacrylate; styrene; 1-vinylimidazole; 2-vinylpyridine; 4-vinylpyridine; divinylbenzene; ethylene glycol dimethacrylate; N,N′-methylenediacrylamide; N,N′-phenylenediacrylamide; 3,5-bis(acryloylamido) benzoic acid; pentaerythritol triacrylate; trimethylolpropane trimethacrylate; pentaerytrithol tetraacrylate; trimethylolpropane ethoxylate (14/3 EO/OR) triacrylate; trimethylolpropane ethoxylate (7/3 EO/OR) triacrylate; trimethylolpropane propoxylate (1 PO/OR) triacrylate; trimethylolpropane propoxylate (2 PO/OR) triacrylate; and mixtures thereof.

A solid substrate should withstand changes in temperature necessary for the methods described herein, as well as enzymatic processes, buffer systems, and repetitive wash steps performed during the method. The substrate may also include paramagnetic components which permit reversible binding to a magnet.

When immobilizing the e.g., target ssDNA sequence to a substrate, the target ssDNA molecules should be spaced sufficiently far from each other on a solid support to prevent ligation of a single probe to two target ssDNA fragments. The distance between each molecule is dependent on the approximate length of each fragment and can vary from 1 to 1000 nm.

### Probe Library

The probe libraries for use in the methods described herein for conversion comprise a double-stranded portion and two single-stranded overhangs. A “probe library” comprises a plurality of distinct oligonucleotide sequences with multiple copies of each distinct oligonucleotide in one mixture. The number of distinct oligonucleotide sequences determines the “complexity” of the library and is determined by the number of random (e.g., degenerate) nucleotides in each probe, such that probes comprising all possible combinations of A, T/U, C and G are accounted for in one library. A probe library may be comprised of molecules which have a single pre-determined code for converting one base per conversion cycle or comprised of molecules with multiple, serially aligned pre-determined codes for converting more than one base per conversion cycle. Pre-determined codes may be designed such that a single codes represents a single base or a single base is represented by more than one code.

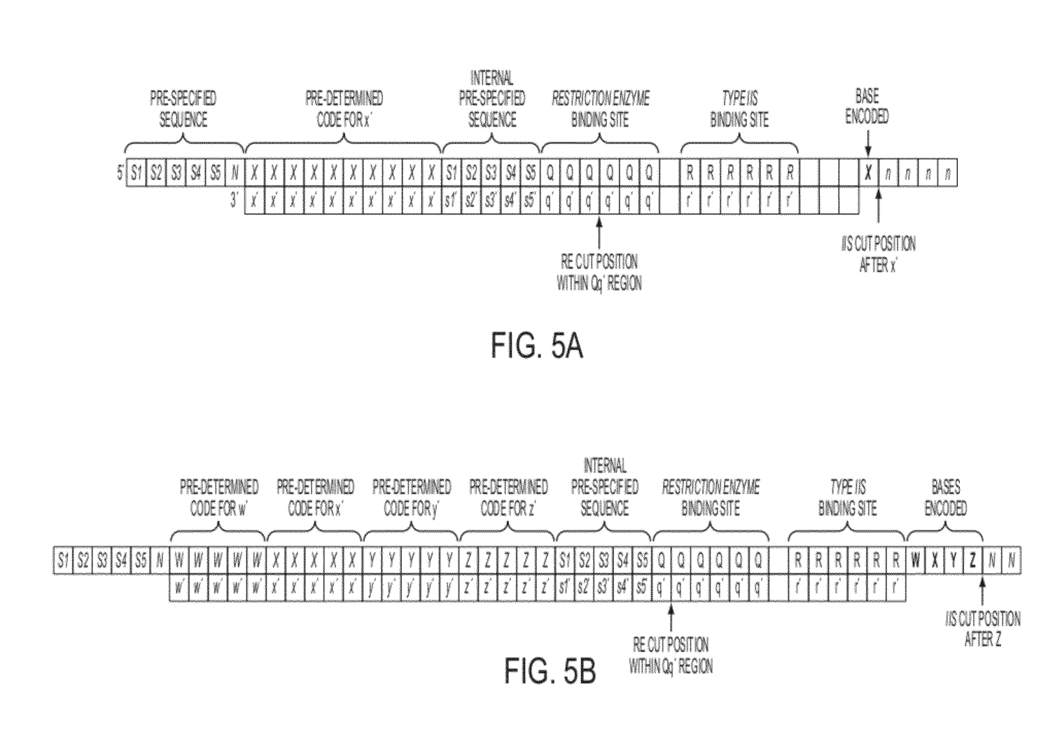

For the purposes of illustration only see FIG. 5A, which depicts an exemplary oligonucleotide probe of the probe library for conversion. The probe described in FIG. 5A is useful for converting a target ssDNA molecule from its 3′ end, however it is also contemplated herein that a target ssDNA molecule is converted from its 5′ end with an analogous probe configuration. Each probe has a double-stranded region, flanked by two single-stranded overhangs. The length and the sequence composition of each end are independently variable but generally will be between 5-12 bases in length. Some of the bases will have a pre-determined order and be comprised of unique bases while other positions may be comprised of a mixture of all four nucleotides at a given position. Increasing the length of a single-stranded overhang increases the stability of the hybrid with the ssDNA target when formed. Use of mixture of bases “N” or “n′” at a given position are generally used to ensure enough sequence variability in the probe library to hybridize to all possible sequences possible in the target ssDNA. “N” or “n′” may also be used to indicate where a base may be varied but represents a unique base in a probe library, e.g., at the end or the pre-specified sequence 5′-S1, S2, S3, S4, S5, N-3′ the “N” is variable depending upon the restriction enzyme used to cleave Qq′ region. Both overhangs are comprised by the same strand (5′-3′; i.e., upper strand) and are thus separated by the 5′-3′ directional strand of the double-stranded portion of the probe. In this example, the nucleotides labeled 5′-S1, S2, S3, S4, S5, N-3′ present on the first single-stranded overhang of the probe form a pre-specified sequence, that is complementary to the pre-specified sequence attached to one end of a target ssDNA molecule, 5′-n′, s5′, s4′, s3′, s2′, s1′-3′. The first single-stranded overhang also comprises at least one pre-determined nucleotide “N”, the nucleotide(s) varies depending upon the cutting specificity of restriction enzyme used to cleave the Q/q′ region, see FIG. 5 for example a “C” is cleaved so N is the complement thus “G”. “N” may be a single pre-determined base or more than one pre-determined bases as necessary. The single-stranded overhang on the 3′ end of the double-stranded portion of the probe comprises at number of added random nucleotides to increase the hybridization efficiency. The number of random bases may be generally from 2-4. When 4 random bases are used there are 256 possible combinations of A, T/U, C, G for a given nucleotide sequence. When single base conversion is performed, the nucleotide immediately adjacent to the double-stranded portion is complementary to the nucleotide of the target ssDNA to be converted, and is designated as bold “X” in FIG. 3A-3E. The length of each overhang can vary from as little as 3 nucleotides to as many as 12 nucleotides. It is important to note that as the length of the overhangs on the probe increase, so does the complexity of the probe library. For example, a probe with a 3′ overhang of 12 nucleotides requires a library with complexity of 4¹² (i.e., 11 degenerate nucleotides plus x on the 3′ overhang). The probe library for conversion (FIG. 5A) has a double-stranded DNA portion comprising: a pre-determined oligonucleotide code (X/x′) for the base from the ssDNA target being converted; a repeat of the pre-determined sequence S/s′ or portion thereof; a recognition sequences Q/q′ for a restriction enzyme binding site which cuts within the Q/q′ region; and a Type IIs restriction enzyme recognition sequence R/r′ which cuts outside of the R/r′ region into the ssDNA. In one embodiment, sequences from defined regions may share base(s) in common.

An exemplary probe library shown in FIG. 5A following conversion to a circular form is contacted with a type IIs restriction enzyme that binds to (R/r′) and cleaves outside of its recognition sequence to the 5′ side of the second single-stranded overhang (FIG. 5). In this example, the first single-stranded overhang comprises the sequence 5′-S1, S2, S3, S4, S5-3′ that is complementary to the sequence in positions 2-6 of the pre-determined oligonucleotide code (5′-s5′, s4′, s3′, s2′, s1′-3′) of the target ssDNA, followed by a position N (N may be a single or more than one nucleotide) that is pre-determined by the restriction enzyme used to cleave R/r′; the second single-stranded overhang (5′-X, n,n,n,n-3′) comprises a sequence that is complementary to the nucleotide to be converted (x′) followed by at least 4 positions that are represented by all four nucleotides in the probe library. FIG. 1 shows one embodiment of a probe hybridizing to a target ssDNA under conditions that permit one of a plurality of probes in the library to form a perfectly matched duplex with a target ssDNA molecule (note that x′=T is the nucleotide to be converted in this example).

The double-stranded portion of the probe comprises a pre-determined known sequence Xx′, designated as x's in the lower, shorter strand, and Xs in the complementary upper, longer strand X, as shown in FIG. 5A. A predetermined sequence of 10 Xs and x's is shown for illustrative purposes. The number of Xs and x's in the sequence can range from 8-25. In one embodiment, the complement of the known pre-determined sequence binds to a specified optically detectable molecule, for example a molecular beacon. The double-stranded portion also contains the sequence S/s′, designated as 3′-s1′, s2′, s3′, s4′, s-5′ in the lower, shorter strand and the compliment S1, S2, S3, S4, S5 in the longer, upper strand. This S/s′ sequence is used to regenerate the pre-specified nucleotide sequence. The double-stranded portion also comprises the sequence Q/q′, which is a 4-9 nucleotide sequence for the recognition and cutting site for a restriction enzyme. The double-stranded portion of the probe further comprises a type IIs restriction enzyme recognition site, R/r′. The IIs site is encoded in a region such that the restriction enzyme recognizes the site and cleaves at least one nucleotide (designated as x′ in FIG. 5A) from the target ssDNA (e.g., the nucleotide to be converted). It is important to consider the cleavage characteristics of both the restriction enzymes. The Type IIs chosen in combination with the upper and lower library strands configures the position of the IIs binding site with respect to the position of the cleavage site such that a desired number of nucleotides are converted in each round. Thus, if four nucleotides are desired to be converted, it is desirable that the IIs cleavage site cleaves such that four terminal nucleotides are removed from one terminus of the target ssDNA molecule each cycle (FIG. 5B). Thus, the position of the recognition site in the probe should be an appropriate distance for the desired enzyme to achieve the correct cleavage site. For example, if the IIs enzyme used is EarI, which cleaves 4 nucleotides downstream of its recognition site on the 3′-5′ strand (i.e., the bottom strand shown in the figures herein), when the IIs recognition site is placed on the end of the double-stranded region 4 bases may be encoded per conversion cycle. If the EarI recognition site is moved upstream of the terminal nucleotide into the double-stranded region by 3 bases, then EarI would cleave a single base from the target ssDNA per conversion cycle. Type IIs restriction enzymes with short distances between their recognition site and their cleavage site (e.g., 2-4 nucleotides) require that the IIs recognition sequence is closer to the 3′ end (using the top, longer strand as reference) of the double-stranded portion of a probe. Conversely IIs restriction enzymes with very long distances between their recognition and cleavage sites require that the recognition site is closer to the 5′ end of the double-stranded portion of the probe and in some cases the length of strands may need to be expanded to ensure that the correct number of nucleotides are present between the recognition and cleavage sites. Thus, the IIs restriction enzyme utilized can affect the length of a probe required for the methods described herein. Type IIs restriction enzymes which cleave 1-4 nucleotides are preferred for the conversion process. The sites Q/q′ and R/r′ may or may not be separated by one or more additional nucleotides to minimize stearic interactions between the 2 restriction enzymes.

The 5′ end of the pre-determined code or codes defined by any of Ww′, Xx′, Yy′, Zz′ further comprises a pre-specified sequence that is complementary to the pre-specified target sequence ligated to the target ssDNA molecule for the first round of conversion (see for example FIG. 5B, wherein the top strand of the probe comprises a 5′-S1, S2, S3, S4, S5, N-3′ sequence wherein the target DNA comprises a 5′-n′, s5′, s4′, s3′, s2′, s1′-3′ sequence). The probe library in the double-stranded region also contains a repeat of the 5′-S1, S2, S3, S4, S5-3′ sequence adjoining the restriction enzyme site defined by Q/q′. This permits binding of a second oligonucleotide library probe in the second round and binding of additional oligonucleotide library probes in each successive conversion round. It is important to note that a bound oligonucleotide is consumed during the process of conversion, thus for each successive round it may be necessary to use a fresh aliquot of the probe library, enzyme mixtures and wash buffers. Probes can be synthesized by any means known to one of skill in the art, (e.g., an oligo synthesizer), or alternatively a probe library can be purchased from a commercial source such as IDT (available on the internet at idtdna.com), Invitrogen (Carlsbad, Calif.), etc.

For the purposes of converting a target ssDNA from either its 5′ end or 3′ end, probes are synthesized with the following changes: (1) the first and second overhangs are interchanged so that the probe is in the correct orientation for converting the desired end, (2) the recognition site sequence R/r′ of the Type IIS restriction enzyme sequence is orientated such that at least one terminal nucleotide on a target ssDNA molecule is cleaved, (3) the type IIs restriction enzyme recognition site is designed such that the appropriate number of nucleotides is present between the recognition site Rr′ and the cleavage site on the target ssDNA for the desired restriction endonuclease, and (4) the restriction enzyme site Q/q′ sequence may or may not be reversed depending upon the specific enzyme used. Many restriction enzymes recognize a palindromic sequence which implies the sequence is not changed by inverting.

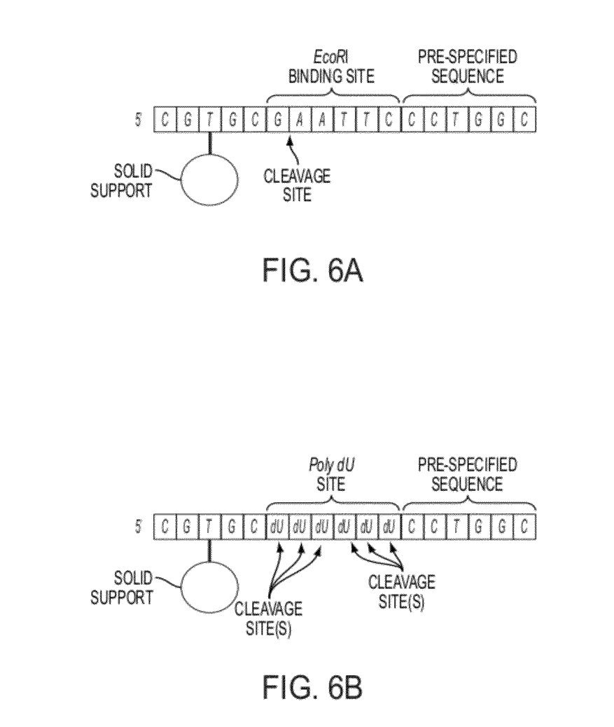

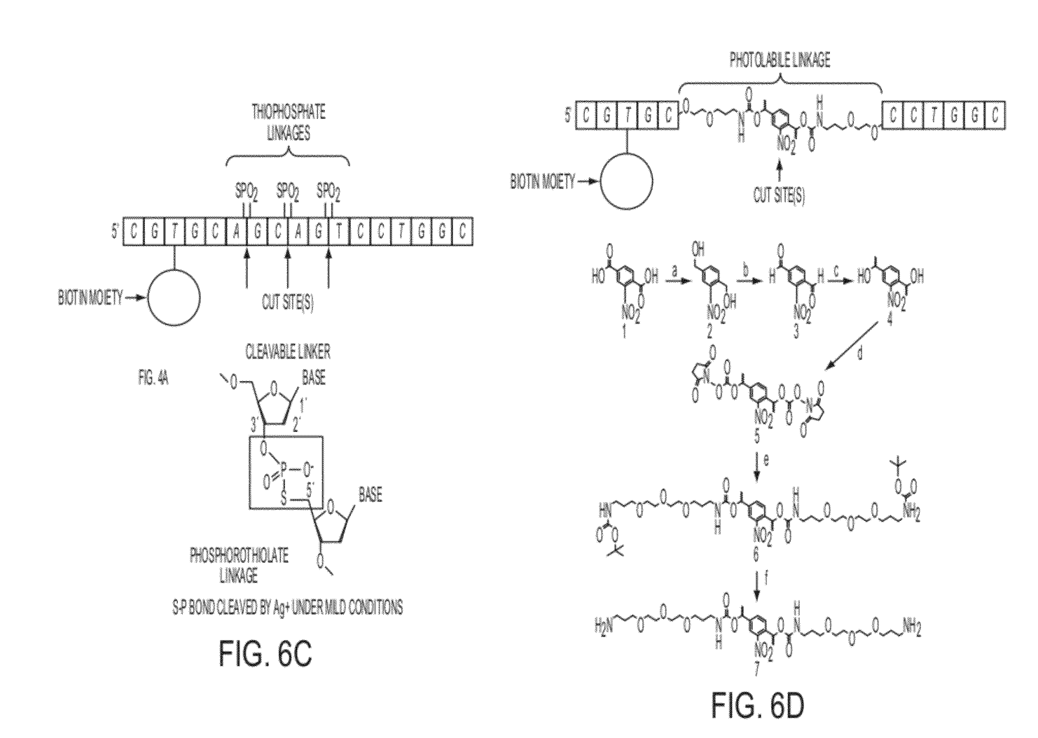

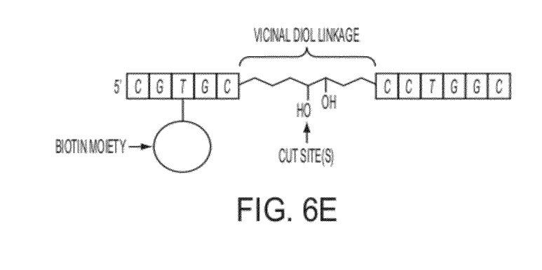

In one embodiment, an additional probe (also referred to herein as an “elution probe”; not shown) is used following conversion and hybridization of the optically detectable molecules such that the converted ssDNA is cleaved off of the structural support e.g., for further nanopore-based sequencing. For example, in one embodiment, the target ssDNA is initially tagged with a pre-specified sequence further comprising a Type II restriction enzyme recognition site (see FIG. 6A); however the single-stranded nature of a converted ssDNA molecule does not permit cleavage using a type II restriction enzyme. Thus, an additional single-stranded probe is used to bind to the targeted cleavage region of a converted ssDNA molecule to complete a double-stranded recognition/cleavage site. Contact with an elution probe and further contacting the system with a desired type II restriction enzyme (e.g., EcoRI) permits cleavage of the target ssDNA molecules from the support for further sequencing as desired. Additional bases may be inserted into the pre-specified sequence to minimize surface-enzyme interactions, for example additional bases of polyT might be inserted on either or both sides of the base containing the biotin.

It should be noted that in addition to the nucleotides A, C, T, and G, nucleotides in the 3′-overhang end of the probe library can be inosine (I) or other nucleotide analogs that indiscriminately pair with guanine, adenine, thymine, uracil or cytosine. In this manner, the complexity of the library can be decreased permitting increased efficiency of conversion. Such positions should not be too close to the ligation site, otherwise they may interfere with the ligation reaction, however it can be as close as the 6th position from the ligation site (i.e., the positions in the upper, longer probe hybridizing to the corresponding position in the target ssDNA can be an inosine). Having multiple inosine positions (e.g., the 6th, 7th, 8th and 9th positions) will not increase the library's complexity but will give a larger footprint for the ligase to work more efficiently.

One aspect of the methods described herein relates to an oligonucleotide probe library comprising T-shaped probes, which are useful for the methods of DNA conversion described herein.

### Ligases

Ligation can be accomplished either enzymatically or chemically. Chemical ligation methods are well known in the art, e.g., Ferris et al, Nucleosides & Nucleotides, 8:407-414 (1989); Shabarova et al, Nucleic Acids Research, 19:4247-4251 (1991); and the like. In some embodiments, however, ligation is carried out enzymatically using a ligase in a standard protocol. Many ligases are known and are suitable for use in the invention, e.g., Lehman, Science, 186:790-797 (1974); Engler et al, DNA Ligases, pages 3-30 in Boyer, editor, The Enzymes, Vol. 15B (Academic Press, New York, 1982). Non-limiting examples of ligases include T4 DNA ligase, T7 DNA ligase, E. coli DNA ligase, Taq ligase, Pfu ligase, and Tth ligase. Protocols for their use are well known, e.g., Sambrook, et al. 1989 “Molecular Cloning: a Laboratory Manual, 2nd Edition, Cold Spring Harbor, N.Y., Cold Spring Harbor Laboratory Press; Barany, PCR Methods and Applications, 1:5-16 (1991); Marsh et al., Strategies, 5:73-76 (1992). Generally, ligases require that a 5′ phosphate group be present for ligation to the 3′ hydroxyl of an abutting strand.

A “ligase” as used herein refers to an enzyme that catalyzes the joining of a sugar phosphate backbone of two nucleic acid sequences. Thus, a ligase joins the backbone of two independent DNA sequences to produce one seamless DNA sequence at that site. Two types of ligases can be utilized for the practice of the methods described herein: (a) DNA ligase (e.g., T4 DNA ligase), and (b) RNA ligase (e.g., T4 RNA ligase).

An RNA ligase (e.g., T4 RNA ligase), which also has activity on single-stranded DNA, can be used to attach a pre-specified sequence tag to one end of a target ssDNA molecule. This sequence tag is used for hybridization with an oligonucleotide probe on the first round of conversion. Since most DNA ligases are active only on double-stranded DNA molecules, the pre-specified sequence can be added to a single-stranded DNA molecule by the use of an RNA ligase. This ligase is also useful for tagging a target RNA molecule for use with the methods described herein. The activity of this enzyme is lower on a single-stranded DNA molecule than the activity on a single-stranded RNA molecule, thus longer incubation times may be necessary for attaching a tag onto a target ssDNA molecule.

In an alternate embodiment, a DNA ligase is used to add the pre-specified nucleotide sequence to one end of a target ssDNA molecule followed by denaturation of the dsDNA to ssDNA, as described herein in the “Target nucleic acid templates” section.

The DNA ligase is also used herein to join one double-stranded DNA fragment with an overhang and one single-stranded DNA fragment together and is useful for ligating an oligonucleotide probe to a target ssDNA molecule. Essentially, the target ssDNA and the oligonucleotide are ligated together to form a continuous circle comprising a double-stranded portion at the probe region. This circle, produced by a ligated oligonucleotide probe and the target ssDNA molecule, is referred to herein as a “reaction circle” or a “target ssDNA/probe circle”.

In general, commercial ligases are derived from T4 bacteriophage or E. coli, however ligases from other sources are also contemplated. In one preferred embodiment, a thermostable ligase, such as Ampligase®, can be used. A thermostable ligase allows ligation under higher stringency temperatures, which can be tailored as necessary to permit specific hybridization of a distinct oligonucleotide probe.

Reaction conditions for commercial ligases can vary and methods for use are supplied by the manufacturer. These methods can be performed by one of skill in the art and changes to the reaction conditions to provide optimal performance of the ligase for the methods described herein are well within the abilities of one skilled in the art.

### Restriction Endonucleases

As used herein, the term “restriction enzyme digestion” of DNA refers to the catalytic cleavage of a DNA sequence with an enzyme that acts only at certain locations in the DNA (i.e., restriction endonucleases), and in general the sites for which each is specific is called a restriction site. The various restriction enzymes contemplated for use herein are commercially available and their reaction conditions, cofactors, and other requirements as established by the enzyme suppliers are used. Appropriate buffers and substrate amounts for particular restriction enzymes are specified by the manufacturer. Incubation for about 1 hour at 37° C. is ordinarily used, but may vary in accordance with the supplier's instructions. Two types of restriction endonucleases are useful in the practice of the methods described herein: (a) Type IIS and (b) Type II restriction endonucleases.

The type IIs restriction enzymes are used for cleavage of the terminal nucleotide(s) of a target ssDNA molecule that is to be converted. Type IIs restriction enzymes (e.g., BtsCI, EarI, FokI, AlwI, MmeI) cleave outside of their recognition sequence to one side. These enzymes recognize sequences that are continuous and asymmetric. This cleavage pattern is achieved by two distinct domains on the enzyme, one for DNA binding, the other for DNA cleavage. They are thought to bind to DNA as monomers for the most part, but to cleave DNA cooperatively, through dimerization of the cleavage domains of adjacent enzyme molecules. An example of a Type IIs restriction enzyme is EarI, that recognizes the asymmetric sequence 5′-CTCTTC/3′-GAGAAG and cleaves 4 nucleotides downstream on the 3′-5′ lower strand, thus potentially removing 4 bases from the ssDNA being converted. Type IIs restriction recognition sites are incorporated into a probe useful in the methods described herein.

Essentially almost any Type IIs restriction enzyme can be used in the methods described herein, including enzymes that leave behind a blunt end. It is important that a restriction enzyme with consistent cleavage properties is used in the methods described herein (e.g., specific cleavage site). In some instances only one nucleotide will be cleaved from the end of a target ssDNA molecule, thus an enzyme that does not cut consistently at its specific cleavage site will cause an error during conversion and any subsequent sequencing. It is also important to consider the length of time that a restriction enzyme takes for substantially complete cleavage. In one embodiment, a type IIs restriction enzyme is chosen that has a relatively short cleavage time, which permits successive rounds to occur in a relatively short time frame (e.g., to speed rate of conversion of a longer target ssDNA template). The type IIs restriction enzyme can be any recognized sequence of any type IIs restriction enzyme as defined by Roberts et al. (2003) Nucleic Acids Research 31:1805-1812, which is incorporated herein by reference in its entirety. In addition, it is contemplated herein that novel type IIs restriction enzymes that are (a) newly discovered in nature, (b) recombinantly produced, or (c) modified, can also be used with the methods described herein.

Some type IIs restriction enzymes are not useful for the methods described herein, and thus a type IIs restriction enzyme should be chosen with care. For example, some type IIs restriction enzymes cleave DNA on both sides of their recognition sequence (e.g., PsrI, PpiI, Hin41, AloI, BsaX, BcgI, CspCI, BaeI) and should be avoided in the methods described herein. It is possible to use these enzymes provided that the end of the target nucleic acid molecule that is not converted does not comprise a complete double-stranded cleavage site.

In addition, some type IIs restriction enzymes have a cleavage site that requires a specific end nucleotide (e.g., Adenine) for cleavage instead of a degenerate nucleotide (e.g., n). Thus, these types of enzymes will only cleave target ssDNA molecules with this specific terminal nucleotide (e.g., Adenine) and therefore any target ssDNA molecules with other terminal nucleotides (e.g., Thymine, Cytosine, Guanidine) are not cleaved. Since the nucleotide sequence of a target ssDNA is unknown, it is not possible to use such enzymes for the process of conversion. Some examples of these enzymes include BsmI, BbvCI, BssSI, BseYI, Bpu10I, which are not contemplated for use herein.

Type II restriction enzymes are used in the methods described herein for the purpose of cleaving a converted ssDNA molecule at site defined by Q/q′ to regenerate the pre-specific sequence S/s′ to enable another probe molecule to join in the next and subsequent cycles. There a numerous commercially available Type II enzymes from which to choose and those chosen are based upon reaction specificity and yield rather than recognition sequence or cut location with the sequence. Some non-limiting type II enzymes are those such as SalI, HhaI, HindIII, BamHI, KpnI, and NotI that cleave DNA within their palindromic recognition sequences. Cleavage leaves a 3′-hydroxyl on one side of each cut and a 5′-phosphate on the other.

Type II restriction enzymes may also be used in the methods described herein for the purpose of releasing a converted ssDNA molecule from its solid support for further sequencing using, for example a nanopore-based technology. It may be desirable to have a different Type II restriction enzyme for this purpose than used to cut within the probe sequence Q/q′ but not a requirement. Some non-limiting type II enzymes are those such as EcoRI, HhaI, HindIII, BamHI and NotI that cleave DNA within their palindromic recognition sequences. Since the Type II restriction recognition site is attached to a target ssDNA molecule along with a pre-specified tag sequence, the region where the target ssDNA molecule is cleaved is consistent among all target ssDNA fragments. The target ssDNA molecule cannot be cleaved by a Type II restriction enzyme until a separate probe is added to complete the (palindromic) double-stranded sequence. An elution probe designed for this purpose is contemplated and discussed herein.

### Hybridization Conditions

Nucleic acid hybridization involves contacting a probe with a target ssDNA under conditions where the probe and its complementary target ssDNA can form stable hybrid duplexes through complementary base pairing. The nucleic acids that do not form hybrid duplexes are then washed away leaving the hybridized oligonucleotides to be used in sequence preserved DNA conversion. Optimal hybridization conditions will vary with the length of probe and the stringency of conditions required for appropriate probe binding. In general, lower temperatures permit a larger number of probes to bind a target ssDNA (including non-specific probes), while higher temperatures permit a smaller number of probes to bind a target ssDNA due to an increase in stringency (e.g., only probes that specifically hybridize are permitted to bind a target ssDNA under stringent conditions).

General hybridization techniques are described in Hames and Higgins (1985) Nucleic Acid Hybridization, A Practical Approach, IRL Press; Gall and Pardue (1969) Proc. Natl. Acad. Sci. USA 63: 378-383; and John et al., (1969) Nature 223: 582-587. Methods of optimizing hybridization conditions are described, e.g., in Tijssen (1993) Laboratory Techniques in Biochemistry and Molecular Biology, Vol. 24: Hybridization With Nucleic Acid Probes, Elsevier, N.Y.). Conditions that promote annealing are known to those of skill in the art for DNA compositions and are described in Sambrook et al., (1989), Molecular Cloning: A Laboratory Manual, Second Edition, Cold Spring Harbor, N.Y.).

### Barcoding

In the high throughput environment of medical genomic analysis there is a benefit to multiplexing analysis from more than one sample by mixing samples from different sources prior to analysis. The concept of barcoding samples is not new and has been used in many formats depending upon the technology used however the mode of barcode implementation in this invention is novel. Barcodes are generally a series of bases, number varying from 3-10 depending upon how many different samples might need tracking via the barcode. The order of bases tracks back to a specific sample.

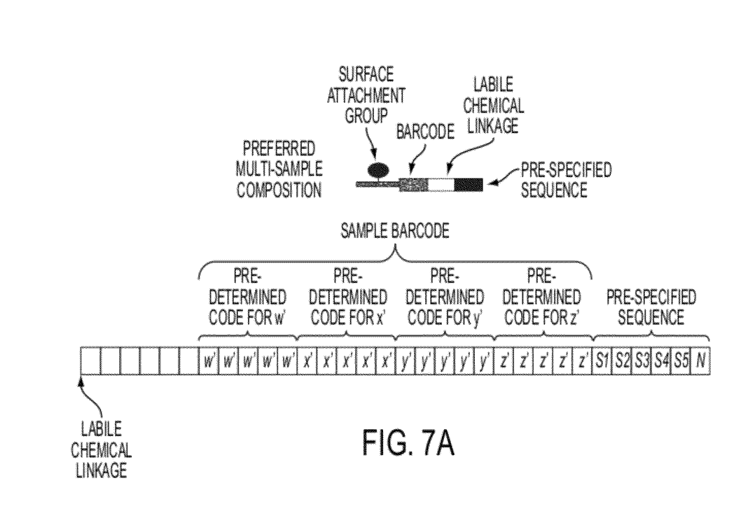

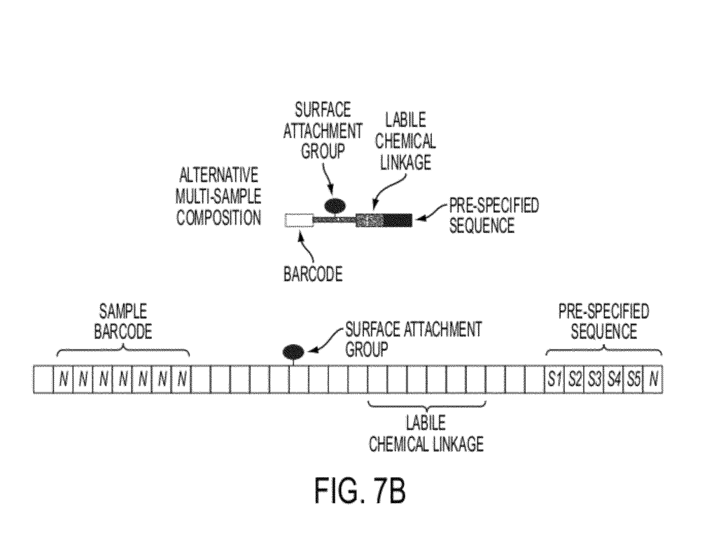

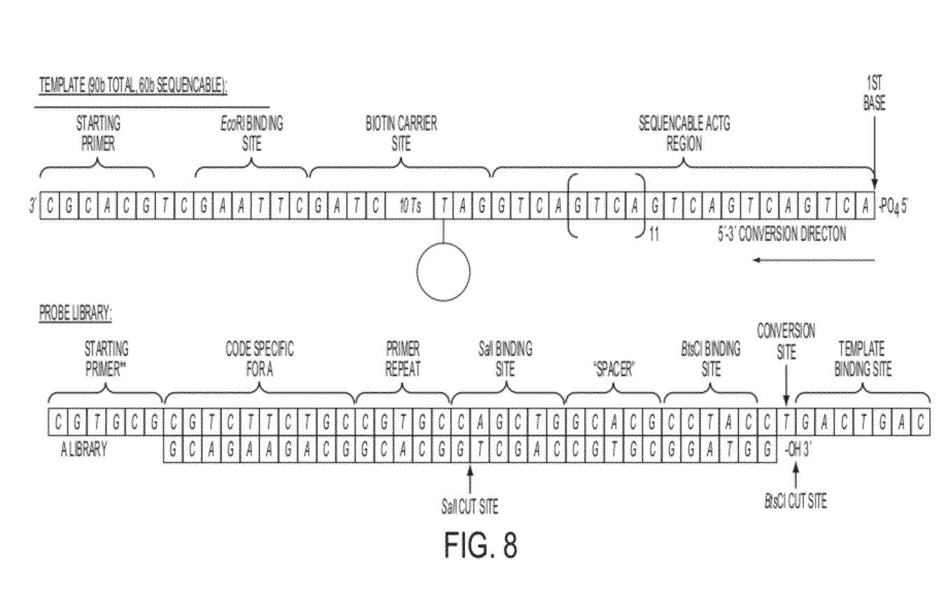

In the specific example of the chemical conversion process, a sequence of bases incorporated into the sample modifier ligated to each target ssDNA incorporates a barcode (FIGS. 7A and 7B). The positioning of the barcode within the oligonucleotide determines how the barcode is utilized. When the bases comprising the barcode are positioned (referenced from converting from the 3′-end of the target ssDNA) between the site of surface attachment and the 5′-end of the target ssDNA, the entire target ssDNA plus the bases used in the barcode must be converted in order to link the sequence with the sample (FIG. 7B). In a preferred configuration, the barcode is positioned between the 3′-end of the target ssDNA and the surface attachment. In this configuration, the position is beyond that converted by the conversion process and therefore the barcode is comprised of the pre-determined codes used for converting the target ssDNA in a specific order comprising from 4-10 codes which defines the specific samples in the mixture. In this configuration the entire target ssDNA does not need to be converted before associating the barcode with the sample, the barcode is linked to the sample even if only a single base is converted (FIG. 7A).

The barcode in either configuration above is also able to be uniquely differentiated by adding an additional level of coding preceding, following or both which defines the boundaries of the sample specific barcode. The number of bases or codes is not restricted, for example, one could use five (5) bases in the sequence “AAAAA”, “GGTAA”, “CCAAA”, or any combination of A, G, C, T as the code which precedes the actual barcode. The same or different sequence may also be found on the other side of the barcode. The codes used may be comprised of individual bases and converted or maybe be comprised of the converted codes depending upon configuration as described above.

Exemplary Method of Converting a Target ssDNA Molecule

A method is described herein for sequentially converting each nucleotide in a target ssDNA molecule into a converted ssDNA molecule, wherein each converted nucleotide is excised and substituted for by a known sequence that represents that nucleotide. The known sequences are essentially a code comprising a pre-determined set of nucleotides that represents each nucleotide. This code can be a binary code. In methods of the invention, the order of the target ssDNA sequence is preserved, however it is the known sequences that are used for further sequencing of the molecule rather than sequencing at single nucleotide resolution. For example, a converted adenine nucleotide is replaced with a 12-mer of known sequence derived from the oligonucleotide probe.

A method is described herein for sequentially converting each nucleotide in a target ssDNA molecule into a converted ssDNA molecule, wherein each converted nucleotide(s) is removed from the target ssDNA following conversion. In one embodiment, the method of conversion comprises the following steps: (a) attaching a sample modifier comprising a moiety for immobilization to a solid support and a pre-specified sequence to a target nucleic acid to provide a modified nucleic acid, (b) immobilizing the modified nucleic acid onto a solid support, (c) ligating a probe library comprising a pre-determined expanded code for one or more bases to the immobilized modified nucleic acid, (d) forming a circular molecule by circularizing the molecule produced in step (c), (e) cleaving the circular molecule with one or more restriction enzymes, (f) optionally separating and washing away the double-stranded portion leaving single-stranded nucleic acid on the solid support, (g) repeating steps (c) to (f) two or more times, wherein the conversion results in a converted molecule which can be used to determine the nucleotide sequence of the target nucleic acid. An exemplary method of conversion is described herein in more detail (FIG. 3A-3E).

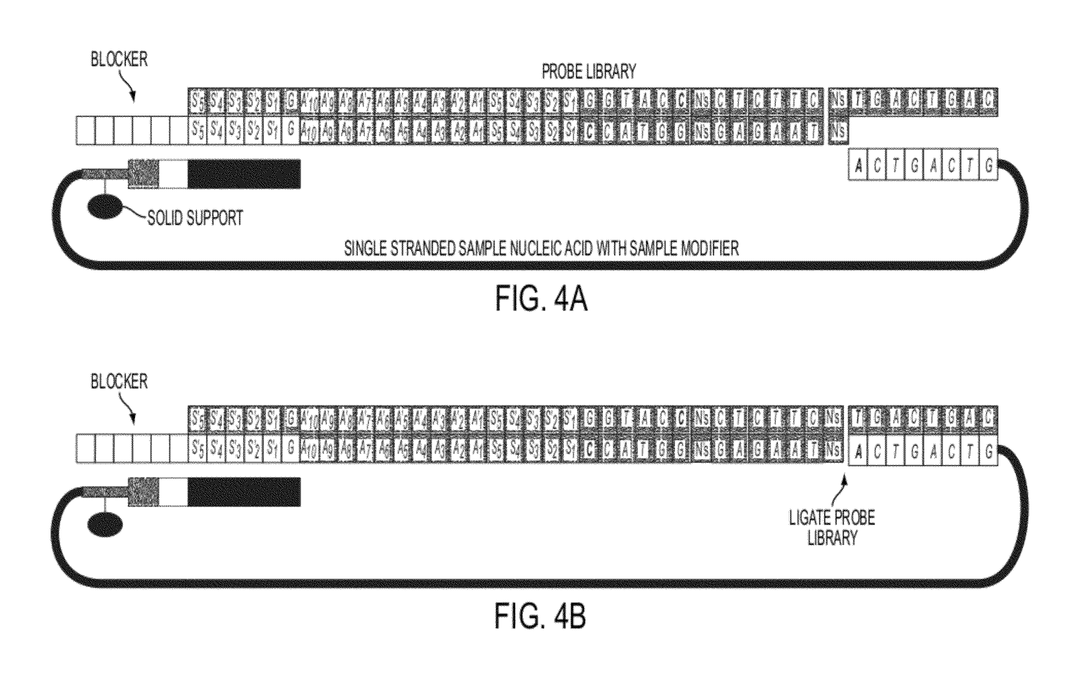

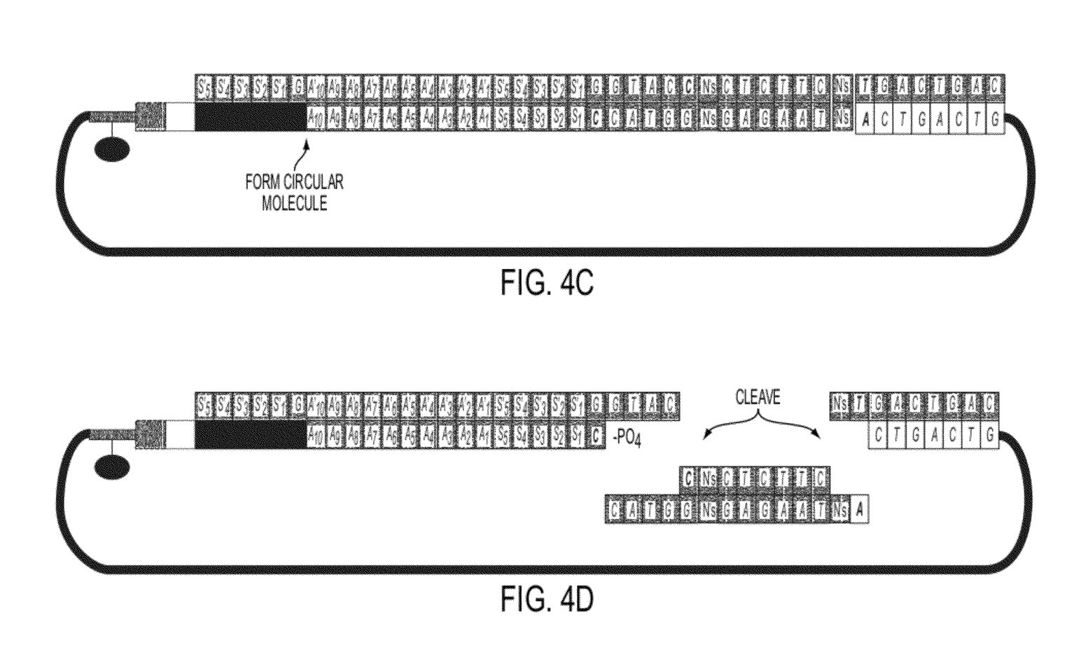

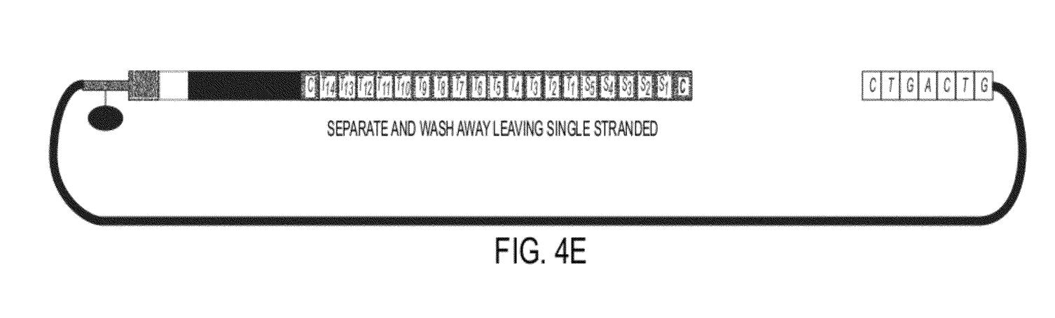

A method is described herein for sequentially converting each nucleotide in a target ssDNA molecule into a converted ssDNA molecule, wherein each converted nucleotide(s) is removed from the target ssDNA following conversion. In one embodiment, the method of conversion comprises the following steps: (a) preparation of a fragmented target template by ligating a sample modifier to one end of the molecule and immobilizing the target ssDNA template onto a solid support, (b) contacting the immobilized target ssDNA molecule with an oligonucleotide probe library comprising a plurality of distinct oligonucleotide probes under conditions permissible for specific hybridization wherein only one end of the probe library molecule is able to hybridize due to the presence of a blocking molecule preventing the pre-specified sequence from hybridizing to the target ssDNA, (c) contacting the hybridized target ssDNA/probe complex with a DNA ligase to form a target ssDNA/probe, (d) removing the blocking molecule and treating the complex with a DNA ligase, optionally including an enzyme which is capable of phosphorylating the 5′ end of oligonucleotides, permitting the pre-specified sequence to hybridize and thus forming a circular molecule, (e) contacting the target ssDNA/probe circle with defined restriction enzymes one of which is a Type IIs restriction enzyme, (f) optionally separating and washing away the double-stranded portion of the bound probe, and (g) repeating steps (b)-(f) as desired. Each of the steps may be separated by an intervening wash step. An exemplary method of conversion is described herein in more detail (FIG. 4A-4E).

For illustration purposes, an exemplary method is described below, wherein a target ssDNA molecule is tagged with a sample modifier with a pre-specified sequence on the 5′ end, and the molecule is converted from the 3′ end. It is also contemplated herein that a target ssDNA molecule can be tagged on the 3′ end and converted from its 5′ end.

For the conversion method, a fragmented and immobilized target single-stranded DNA molecule is contacted with a probe library under conditions that permit specific hybridization of a distinct probe (e.g., of 4⁷ distinct probes in a probe library, there will be one that specifically hybridizes to a target ssDNA; of that one distinct probe there can be e.g., thousands of copies). It is preferred that the overhang regions of a distinct probe specifically hybridize with 100% complementarity to a target ssDNA molecule to be converted. The shorter strand of the probe library to be joined to the target ssDNA is in some embodiments phosphorylated on the 5′-end. The hybridization mixture containing the probe library also includes a ligase, e.g., a DNA ligase, which joins one end of the hybridized probe library molecule to the target ssDNA. The solution may contain a blocking molecule to prevent hybridization of the other end of the probe molecule with the other end of the target ssDNA, e.g., the pre-specified sequence region.

Following first ligation, excess probes that are not bound to a target ssDNA molecule are washed away with an appropriate wash buffer. In general, wash buffers comprise a buffered saline solution of a specific pH with an optimal detergent or salt component. Wash buffers with higher salt or detergent concentrations improve the stringency of washes and will remove non-specifically bound probes. The pH can also be raised or lowered to alter the wash stringency. Optimal conditions will vary with a particular wash buffer and is well within the ability of those skilled in the art to prepare and modify such a wash buffer.

The immobilized target ssDNA is then treated in such a way as to remove the blocker molecule, e.g., heat to denature if the blocker is an oligonucleotide or analog thereof and the mixture subjected again to washing conditions to remove unbound blocker. The temperature is then lowered to allow favorable conditions to form a hybrid between the “other end” of the probe, e.g., the pre-specified sequence region, and ssDNA that were not ligated in the prior ligation and contacted again with a ligase under conditions permissible for the ligation (second ligation) of a specifically hybridized probe to a target ssDNA such that a circle is formed, wherein the probe acts as a bridge between the two ends of a target ssDNA molecule. FIGS. 3C and 4C depict an example of ligating both ends of the shorter strand of the bound probe to the target ssDNA with a ligase, thus forming a circular molecule.

Optionally or in combination with using a blocker molecule, the probe library molecules may not comprise a 5′ terminal phosphate required for second ligation of the “other end” of the shorter probe library strand to the target ssDNA forming a circular molecule. Following the initial ligation and removal of the excess library, the immobilized target ssDNA is then treated in such a way as to add a 5′-end phosphate, e.g., use of polynucleotide kinase (PNK) and ATP, using standard methods. The treatment with PNK/ATP may be done as a separate step or in combination with a ligase.

Following second ligation, the ligation mixture is removed and is followed by a wash. The immobilized target ssDNA/probe circle is contacted with a Type IIs restriction enzyme (e.g., EarI, BtsCI), which corresponds to the Type IIs restriction enzyme recognition site R/r′ on the double-stranded portion of a probe. The restriction enzyme cuts at a position several nucleotides away from its recognition site, such that at least one nucleotide (designated as x′ in FIG. 5A) is cleaved off of the 3′ end of the target ssDNA. The target ssDNA/probe complex is linearized during this process and a new 3′ end nucleotide is exposed.

The immobilized target ssDNA/probe complex is also contacted with a Type II restriction enzyme (e.g., SalI), which corresponds to the restriction enzyme recognition site Q/q′ on the double-stranded portion of the probe. The Type II restriction enzyme cuts within the sequence Q/q′ producing a double-stranded fragment defined by the specific cut location within Q/q′ extending to the cleavage site of the Type IIs enzyme. The Type II enzyme may leave 0, 1, or more q′ bases extending from the internal pre-specified sequence “s5′, s4′, s3′, s2′, s1′”.

In a preferred process, the treatment of the target ssDNA/probe complex is treated in a single step with the Type IIs and Type II restriction enzymes combined.

FIG. 5A shows one embodiment of a cleavage step, wherein the ligated molecule is contacted with a type IIs restriction enzyme, e.g., EarI, that specifically recognizes the sequence (R′/R) present in the double-stranded DNA portion of the probe, wherein the enzyme cleaves at least one nucleotide on the 3′ end of the target ssDNA to be converted, thereby removing the converted nucleotide from the 3′ end of the target ssDNA molecule.

In order to remove the remaining unattached, unligated bound probe and return the complex to a single-stranded molecule, the system is treated with ˜0.5N sodium hydroxide and washed. Fragments of the longer strand of the probe are separated from the target ssDNA by denaturing in sodium hydroxide and washed away; thus a probe cannot be re-used. Treatment as described does not disrupt the biotin:streptavidin interaction. One round of the conversion method is now complete. The 3′ end converted target ssDNA molecule in this example comprises on its' 5′ end: the x′ code, the 5′-S1, S2, S3, S4, S5-3′ pre-specified sequence, and the remaining q′ sequence from the oligonucleotide probe. The converted ssDNA is now ready for further conversion cycles.

The system can now be used for further rounds of conversion as desired. The second round proceeds similarly to the first. It is important to note that fresh solution aliquots are used for each successive round, for example a fresh aliquot of probes is used during the hybridization stage. In the second and subsequent repetitive rounds, an oligonucleotide probe distinct for the newly exposed 3′ and 5′ ends of a target ssDNA binds in a manner similar to the first probe. The first single-stranded overhang binds to its complement on the 3′ end of the target ssDNA and the second single-stranded overhang binds to the 5′ end of the target ssDNA. The system is incubated twice under conditions useful for hybridization/ligation with a double-stranded DNA ligase (e.g., T4 DNA ligase) to form a target ssDNA/probe circle. The system is washed, and contacted with a Type IIs restriction enzyme (e.g., BtsCI or EarI) and a Type II restriction enzyme (e.g., SalI) to linearize the molecule and cleave off the endmost 3′ nucleotide(s) of the ssDNA target. The system is treated with base to denature the double-stranded region and the second round is complete. Further rounds proceed in a similar manner until the length of the conversion of essentially all of a target ssDNA fragment is complete (or conversion of a desired portion of a target ssDNA molecule is essentially complete).

It is also contemplated herein that multiple nucleotides are converted at the same time (e.g., at least 2, at least 3, at least 4, or more nucleotides are converted at one time). This does not change the complexity of the library, but will reduce the number of cycles for a given target. The restriction enzyme recognition site, Q/q, is moved the required distance from the cleavage site to permit cleavage of the desired number of nucleotides during each round of conversion (FIG. 5B). Additional pre-determined codes (e.g., W/w′, X/x′, Y/y′, Z/z′) are inserted into the probe library as needed to code for the desired number of nucleotides converted from the target ssDNA.

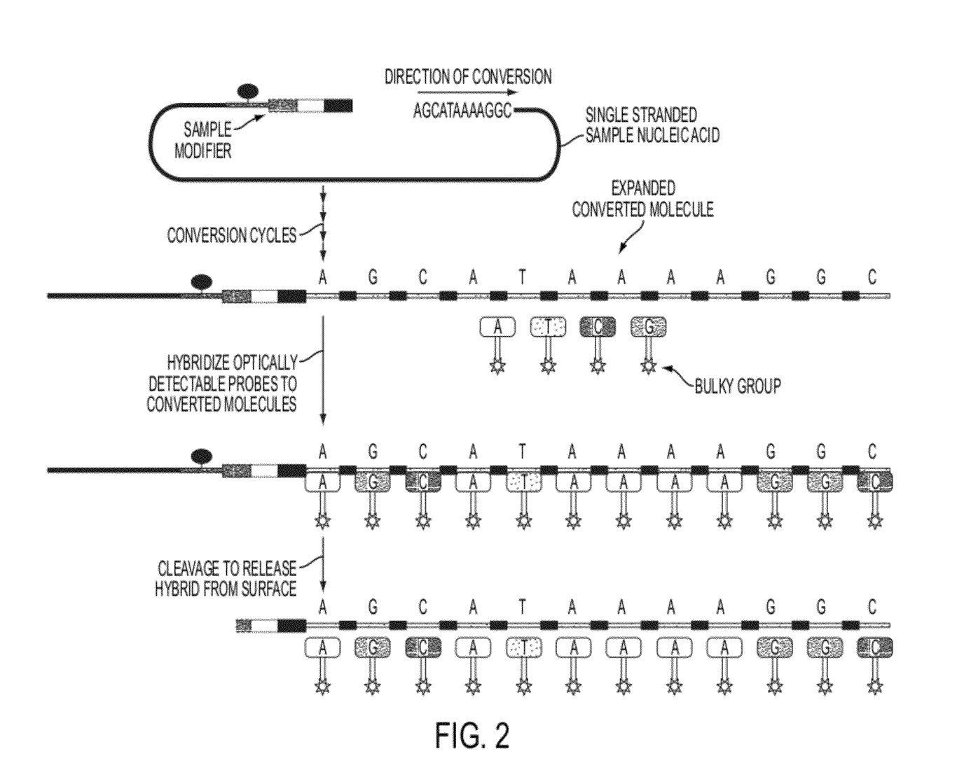

Following conversion, the converted target ssDNA is hybridized to optically detectable oligonucleotide probes (FIG. 2). The detectable probes hybridize specifically to the codes, i.e. w′, x′, y′, z′, for each of the coded bases A, C, G, T or U. The detectable probes have attached fluorophores which are optically resolvable when imaged in a digital imaging system capable of detecting single fluorophores. The preferred configuration is for the detectable probes to have a quencher molecule. Additionally, the probe sequence is that of a molecular beacon, e.g., the probe forms a hairpin structure bringing the quencher and fluorophore into close proximity. The quencher moiety serves two purposes: the primary purpose is to quench or diminish the optical signal from the fluorophore hybridizing on the nearest neighbor code on the converted ssDNA before stripping on the nanopore, and the secondary purpose is to quench the fluorophore on the same code after stripping on the nanopore. Additionally, the probes have a moiety which functions as a bulky group which is larger in diameter than the average diameter of a nanopore to assist in stripping of the detectable probe as the converted ssDNA translocates through the nanopore.

After hybridization of the detectable oligonucleotide probes, the excess probes are washed away and the detector probe/converted target ssDNA complex is released from the support (FIG. 2). Release is in some embodiments performed at a chemically labile site designed within the oligonucleotide comprising the pre-specified sequence and surface attachment moiety, e.g., biotin, which was originally ligated to each target ssDNA (FIGS. 6A-6E). The preferred labile moiety is restriction enzyme binding/cleavage site located between the surface attachment moiety and the pre-specified sequence (FIG. 6A). The restriction enzyme is a Type II, e.g., EcoRI, which binds and cleaves within a palindromic sequence. Since the site is single-stranded in the converted ssDNA, an oligonucleotide which spans the enzyme recognition sequence and having 4-10 additional nucleotides on either side is hybridized to the support bound converted target ssDNA. The restriction enzyme is added in appropriate buffers and the detector probe/converted target ssDNA is release into solution ready for nanopore sequencing.

The methods described herein are useful for converting, and subsequently sequencing, a target ssDNA molecule in less than one week. In one embodiment, the methods described herein can convert a target ssDNA molecule in less than one day (e.g., 16 hours, 12 hours, 8 hours, 4 hours, 2 hours, 1 hour, 30 minutes, 15 minutes, or any integer in-between).

### Nanopore Sequencing

In one embodiment, a converted single-stranded nucleic acid is probed with a nanopore to permit rapid sequencing. The concept of the nanopore-optical readout platform is described in detail in McNally et al., Nano Letters (2010) 10:2237-2244 which is incorporated herein by reference in its entirety. A target ssDNA is biochemically converted to a binary code, wherein each base in the original target ssDNA sequence is represented by a unique combination of 2 binary code units (0 and 1 labeled in open and solid circles, respectively). The converted target ssDNA is hybridized with 2 types of molecular beacons complementary to the 2 code units.

Molecular beacons are hairpin shaped molecules with an internally quenched fluorophore, whose fluorescence is restored when they bind to a complementary target nucleic acid sequence. The use of DNA hairpins as “molecular beacons” (Broude, “Stemloop Oligonucleotides: a Robust Tool for Molecular Biology and Biotechnology,” Trends Biotechnol. 20:249-256 (2002)), either in solution (Tyagi et al., “Molecular Beacons: Probes that Fluoresce upon Hybridization,” Nature Biotech. 19:365-370 (2001); Dubertret et al., “Single-mismatch Detection Using Gold-quenched Fluorescent Oligonucleotides,” Nature Biotech. 19:365-370 (2001)) or immobilized on a solid surface (Fang et al., “Designing a Novel Molecular Bacon for Surface-Immobilized DNA Hybridization Studies,” J. Am. Chem. Soc. 121:2921-2922 (1999); Wang et al., “Label Free Hybridization Detection of Single Nucleotide Mismatch by Immobilization of Molecular Beacons on Agarose Film,” Nucleic Acids. Res. 30:61 (2002); Du et al., “Hybridization-based Unquenching of DNA Hairpins on Au Surfaces: Prototypical “Molecular Beacon” Biosensors,” J. Am. Chem. Soc. 125:4012:4013 (2003); Fan et al., “Electrochemical Interrogation of Conformational Changes as a Reagentless Method for the Sequence-specific Detection of DNA,” Proc. Natl. Acad. Sci. USA 100:9134-9137 (2003)), has proven to be a useful method for “label-free” detection of DNA fragments. Molecular beacons consist of DNA hairpins functionalized at one terminus with a fluorophore and at the other terminus with a quencher. In the absence of their complement, they exist in a closed, “dark” conformation. Hybridization occurs upon introduction of complementary oligonucleotides, which concomitantly forces open the hairpin and allows for a fluorescent, “bright” state.

Each of the beacons used in a nanopore-based sequencing method comprises a fluorophore on its 5′ end and a quencher at its 3′ end or vice versa, with each set of beacons comprising a distinguishing fluorophore (e.g., those that bind the 0 configuration, and those that bind the 1 configuration of the binary code comprise a distinct fluorophore). The broad spectrum quencher molecule quenches both fluorophores, e.g., the quencher molecule prevents fluorescence of the stem loop molecular beacon or it can hinder fluorescence of a neighboring molecular beacon even if the molecular beacon is bound to its complement. The 2 different color fluorophores make it possible to distinguish between the 2 beacons.

Generally, in solution the molecular beacons self-quench and upon hybridization to their targets, molecular beacons are designed to “light up” (Tyagi et al., Nature Biotech 1996; 14:303-8; Bonnet et al., Proc Natl Acad Sci USA 1999; 96:6171-66). However, in the nanopore-based sequencing method, molecular beacons are arranged such that the beacons are next to each other so that quenchers on neighboring beacons will quench the fluorescence emission of its neighboring beacon and the DNA will stay “dark” until individual code units are sequentially removed from the DNA (excluding the first beacon). This concept is a feature of the nanopore-optical readout method; it significantly reduces the fluorescence background from neighboring molecules and from free beacons in solution, resulting in a higher signal-to-background ratio (see U.S. Pat. No. 7,972,858). When the molecule is introduced to the nanopore, the beacons are stripped off sequentially one by one with a time delay of approximately 5-10 ms. This time is tuned by the electric field intensity to optimize the signal-to-background levels (Mathé et al., Biophys J 2004; 87:3205-12; McNally et al., Nano Letters 2008; 8:3418-3422). For example, each time a new beacon is removed, a new fluorophore is unquenched and registered by a custom-made optical detection microscope. By design, the released beacon is automatically closed, quenching its own fluorescence, whereupon it diffuses away from the vicinity of the pore. Immediately upon the release of the 1st beacon, its neighboring beacon's fluorophore will light up. The readout time is estimated (for a single pore) to be in the range of approximately 1 ms/1 base to 10 ms/base, or 100 units/s to 1000 units/s or any point between, for example 2, 3, 4, 5, 6, 7, 8, 9 or 10 ms/base or 150, 200, 250, 300, 350, 400, 500, 600, 750, 800, 900 or 1000 units/s.

In some embodiments, the molecular beacons may be attached to another molecule or chemical, which leads to an increased size and the diameter of the nanopore can be greater than 2 nm, as long as it is small enough to remove the molecular beacons and the attached molecule or chemical, while permitting the converted ssDNA to pass through.

When optically detectable oligonucleotides are not configured as molecular beacons, the fluorophore on the probe released during translocation through the nanopore can be quenched using laser induced photo bleaching which will lower noise in the detection area. Laser induced photo bleaching can be stimulated by using higher power on the laser and/or eliminating chemicals in the detection solution which stabilize fluorophores during excitation, e.g., trolox and dithiothreitol have been used to reduce photo bleaching rates of dyes.

### Sequence Assembly of DNA Fragments

“Sequence assembly” refers to aligning and merging many fragments of a much longer DNA sequence in order to reconstruct the original sequence. Once the signal information has been accumulated through nanopore sequencing, a computer program can be used to assemble the sequence pieces into the original sequence of the target ssDNA molecule. Since the fragmentation of the template is random and independent for each genomic DNA molecule, the sequences of fragments from various genomic DNA molecules overlap. These overlapping regions can be added together using computational software, which analyzes the sequencing results for each fragment, detects overlapping regions between fragments derived from a region of genomic DNA and provides a highly probable sequence for the genomic DNA from the obtained sample.

Computational software for assembling or reconstructing a sequence from fragments can be obtained from a variety of sources. Some examples of DNA assembly software available on the worldwide web for use or purchase include, but are not limited to, Sequencer (genecodes.com), DNA baser aligner (dnabaser.com), CAP3 (pbil.univ_Iyon1.fr.cap3.php; Huang, X. and Madan A. (1999) CAP3: A DNA sequence assembly program Genome Research 9:868-877), AMOS (jcvs.org/cms/research/software/#c614), TIGR assembler (jcvs.org/cms/research/software/#c614), Celera assembler (jcvs.org/cms/research/software/#c614), Phrap (phrap.org) or Clc bio Advanced contig assembly (cicbio.com). Methods for DNA sequence assembly from fragments are known to those of skill in the art.

### Sequencing Automation

In some embodiments, the process of conversion is performed using an automated system that can perform the wash steps, incubation steps and changes in temperature necessary for the conversion methods (e.g., an automated system can inject solutions, permit multiple conversion steps to be performed quickly, reduce contamination from outside DNA sources, and alter temperatures as entered e.g., into a computer program by a user). The system can include such components as a computer, an information storage device, robotic components, a temperature cycler, a microinjection system, buffer and enzyme solution storage etc. This type of system can be designed and used by one of skill in the art, and such a system is contemplated for use with the methods described herein.

In some embodiments, kits are envisioned which will be commercially available for use in either manual or automated systems for optimally performing the chemical conversion. The kits would have the components, formulations and instructions configured in one or more ways for performing the conversion process, i.e., 3′ versus 5′-end sequencing of the target ssDNA, 3′ versus 5′-end sequencing of the target RNA, or sequencing of from 1 to 4 bases per conversion cycle. Included in the kit would be the enzymes (ligase(s), PNK, restriction enzymes both Type II and IIs), enzyme buffers, wash solutions, oligonucleotide probe library, blockers, and optically detectable oligonucleotide probes. Additional kits or kit components which include isolation and preparation of the target ssDNA or surface attachment (streptavidin coated magnetic beads or micro plates) may be included.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### INCORPORATION BY REFERENCE

The entire disclosure of each of the patent documents, including certificates of correction, patent application documents, scientific articles, governmental reports, websites, accession numbers and other references referred to herein is incorporated by reference in its entirety for all purposes.

### EQUIVALENTS

The invention can be embodied in other specific forms without departing from the spirit or essential characteristics thereof. The foregoing embodiments are therefore to be considered in all respects illustrative rather than limiting on the invention described herein.

### EXAMPLES

### Example 1

### Circular DNA Conversion (CDC)

### Conversion of a Target ssDNA Target Molecule Starting at its 5′ End

In this example the conversion process involves a series of hybridization, enzyme incubations and washes all performing on magnetic beads. All the components are commercially available from a variety of sources. Suspend 100 ul of magnetic beads (InVitrogen C1 streptavidin) in a vial. Use magnet to hold beads, and then remove solution. Resuspend beads in 99 ul of bead immobilization buffer (2M NaCl, 2 mM EDTA, 20 mM TRIS, 0.1% Tween). Add 1 ul of biotinylated ssDNA template molecule (Stock conc: 0.5 uM). Leave for 10 mins to allow templates to bind to magnetic beads Use magnet to hold beads, and removed solution. Wash beads 2× with 100 ul Bead immobilization buffer, followed by 2× with 100 ul deionized water. Add 3 ul of probe library (Stock conc: 0.5 uM), 7 ul of Hybridization buffer (50 mM NaCl, 5 mM MgCl₂, 10 mM TRIS, 0.1% Tween). Add 7 ul of deionized water, 2 ul of T4 DNA ligation buffer (10× with ATP, NEB), 1 ul of T4 DNA ligase (50 units, NEB). Incubate for 20 mins @21° C. Use magnet to hold beads, and wash beads 4× with 100 ul Bead immobilization buffer. Resuspend beads in 100 ul of deionized water and incubate for 20 mins @65° C. (heat wash). Wash beads once with deionized water. Resuspend beads in 17.5 ul of deionized water, 2 ul of T4 DNA ligation buffer (10× with ATP), 0.5 ul of PNK (10 units/ul, NEB). Incubate sample for 25 mins @37° C. Use magnet to hold beads, and wash beads 4× with 100 ul Bead immobilization buffer. Resuspend beads in 100 ul of deionized water and incubate for 20 mins @65° C. (heat wash). Wash beads once with deionized water. Resuspend beads in 10 ul of Hybridization buffer, 7 ul of deionized water, 2 ul of T4 DNA ligation buffer (10× with ATP), 1 ul of T4 DNA ligase (50 units). Incubate for 20 mins @21° C. Use magnet to hold beads, and wash beads 4× with 100 ul Bead immobilization buffer. Resuspend beads in 100 ul of deionized water and incubate for 20 mins @65° C. (heat wash). Wash beads once with deionized water. Resuspend the beads in 2 ul of NEB Buffer 4, 2 ul of Sal1-HF (100 units/ul), 2 ul of BtsC1 (20 units/ul), 14 ul of deionized water. Incubate for 30 mins @50° C. Use magnet to hold beads, and wash beads 4× with 100 ul Bead immobilization buffer. Wash beads 2× with deionized water. Resuspend beads in 100 ul of 0.15M NaOH. Leave for 5 mins. Use magnet to hold beads, and wash beads 4× with 100 ul Bead immobilization buffer. Wash beads 2× with deionized water. This completes the first cycle. Samples are now ready for 2^{nd} cycle.

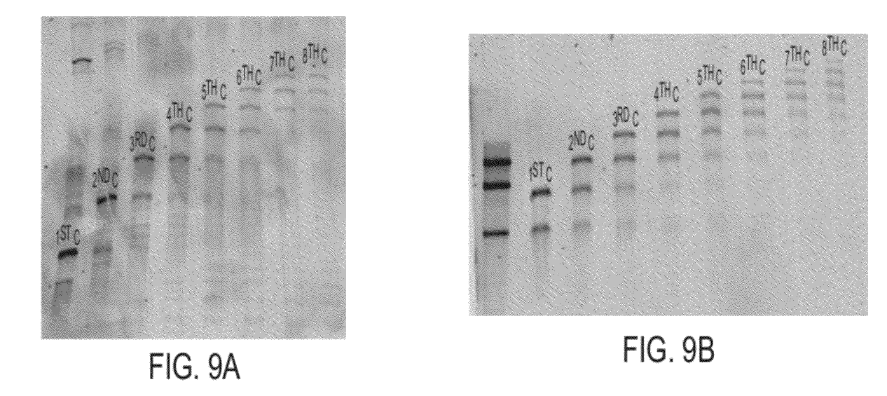

FIGS. 9A and 9B show the products of the first 8 cycles of the conversion process. In FIG. 9A, samples were removed after each of 8 cycles wherein the circular DNA intermediate was formed. The product was separated on an electrophoresis polyacrylamide gel, stained and imaged. IN each lane the expected circular product is identified. IN FIG. 9B, were removed after each of 8 cycles following the restriction digestion with the TypeII/Type IIs restriction enzymes. As with the left image, the expected linear products are identified. Each gel shows that even with reactions not going to completion, the products left behind from one cycle are converted during the next cycle producing the ladder effect seen.

### Example 2

### Formation of Kilobase Circular Single-Stranded DNA

In this example (FIGS. 10A and 10B), 7.4 kB single-stranded, circular M13 phage DNA (M13mp18 cloning vector, NEB) was first digested with 2 restriction enzymes. The restriction enzymes used were BsrGI and AlwNI both available from NEB. Any combination of restriction enzymes might be used to generate fragments of lengths of interest. In order to cut M13 DNA the restriction sites must first be activated by making double-stranded, for this purpose synthetic oligonucleotides were used (20-30 bases) which spanned the restriction enzyme recognition site(s) of interest. For BsrGI the sequenced used was: 5′-AGATGAACGGTGTACAGACCAGGCGC (SEQ ID NO: 2) and for the AlwNI: 5′-ATGGAAAGCGCAGTCTCTGAATTTACCGT (SEQ ID NO: 3). M13 mp18 (10-30 ug) was mixed into NEB Buffer 4, 1.5 molar equivalent of each of the oligonucleotide was added, the mixture heated to 95° C. and cooled to 37° C., approximately 100-200 units of each enzyme added. The digestion reaction was allowed to proceed for 4 hours, the enzymes inactivated at 95° C. for 5-10 min., and digestion mixture used without additional. This combination of restriction enzymes produces 2 ssDNA fragments approximately 1,165 and 6,084 bases in length. Following digestion, the mixture was treated with shrimp alkaline phosphatase (NEB following manufacturer protocol) to remove any 5′-phosphates on the M13 fragments to prevent re-ligation during the subsequent steps.

FIG. 10A describes the strategy used to circularize the fragments. To form circles, two oligonucleotides were designed which when hybridized together simulate the structure of the CDC probe library: a longer top strand with ends complementary to the ends of the M13 fragment of interest for circle formation, and a shorter bottom strand which will be the strand inserted into the M13 piece when joined via two ligations steps. For studies which have steps in which it is desirable to use a surface attachment, a biotin moiety was included during the synthesis of the shorter oligonucleotide strand. For the example shown, the BsrGI:AlwNI 1.1 Kb fragment used as the top, longer strand an oligonucleotide with the sequence: 5′-GTGTACACTGACTGACTGACTGACTGACTGTCTCTGAA (SEQ ID NO: 4) and the shorter, lower strand 5′-CAGTCAGTCAG/iBiodT/CAGTCAGTCAGT (SEQ ID NO: 5). The lower strand was first 5′-phosphorylated using ATP and PNK (5 U, 37° C., 15 min., NEB), PNK heat inactivated, and then hybridized to the top strand (the lower strand was in 1.1 fold molar excess). The hybridized library was then added to an aliquot of the M13 digestion reaction so that the library was in 1.5 molar excess over M13 1.1 kB strand. The reaction mixture in 1× ligation buffer with T4 DNA ligase (40 U, 37° C., 15 min., NEB) was incubated joining the probe library to the ssDNA 1.1 kB fragment. The reaction mixture was added fresh PNK (10 U, 37° C., 15 min., NEB), to phosphorylate the 5′-end of the M13 fragments. T4 DNA ligase was added (40 U, 37° C., 15 min., NEB), to join the other end of the lower strand to the M13 fragment thus forming a 1.1 kB circular ssDNA. An aliquot of the reaction mixture was then heated and snap-cooled to remove any hybridized oligonucleotides forming dsDNA. Exonuclease I (10 U, 37° C., 45 min., NEB) which degrades any single-stranded DNA from its 3′-end was added. Any remaining (circular) DNA showing a band during analysis on an agarose gel (Lonza, FlashGel system, 1.2% RNA gel) would be an indication that the process produced the desired targeted circular DNA. Arrows in FIG. 10B point to the band of circular DNA formed. Additionally, an aliquot of the reaction was added to streptavidin coated beads (M280, Life Tech) and the circular DNA with the biotin inserted was captured and released from the beads for analysis.

What is claimed is:

1. A method for conversion of a target nucleic acid comprising:
(a) attaching a sample modifier comprising a moiety for immobilization to a solid support and a pre-specified sequence to a target nucleic acid, to provide a modified nucleic acid;
(b) immobilizing the modified nucleic acid onto a solid support;
(c) ligating a probe library comprising a pre-determined expanded code for one or more bases to the immobilized modified nucleic acid;
(d) forming a circular molecule by circularizing the molecule produced in step (c);
(e) cleaving the circular molecule with one or more restriction enzymes; and
(f) repeating steps (c) to (e) two or more times,
wherein the method results in a converted molecule which can be used to determine the nucleotide sequence of the target nucleic acid.

2. The method of claim 1, wherein the target nucleic acid is DNA or RNA.

3. The method of any one of claims 1-2, wherein the moiety for immobilization to a solid support is a biotin moiety.

4. The method of any one of claims 1-3, the sample modifier is attached to the target nucleic acid using a ligase.

5. The method of any one of claims 1-4, wherein the sample modifier comprises a barcode, a cleavage site, or a tag sequence.

6. The method of claim 5, wherein the cleavage site is a substrate for an enzyme or a chemical.

7. The method of claim 5, wherein the barcode identifies the sample of origin and is formed by the arrangements of the pre-determined expanded base codes forming a barcode 4-10 codes in length.

8. The method of claim 5, wherein the tag sequence identifies the 5′ end or the 3′ end of the converted molecule.

9. The method of any one of claims 1-8, wherein the solid support is a magnetic particle, polymeric microsphere, or a filter material.

10. The method of any one of claims 1-9, wherein the probe library comprises a plurality of distinct oligonucleotide sequences, each of which includes a double-stranded region, wherein the double-stranded region comprises: two restriction enzyme binding sites, the pre-specified nucleotide sequence, one or more pre-determined codes for each of the bases found in the target nucleic acid, and a first and a second single-stranded overhang, wherein the first single-stranded overhang is a complement to the pre-specified nucleotide sequence and the second single-stranded overhang comprises a plurality of sequences able to complement the target nucleic sequence.

11. The method of claim 10, wherein the pre-determined base codes for each base bind to a molecular beacon.

12. The method of claim 11, wherein there are four pre-determined base codes.

13. The method of any one of claims 1-12, wherein forming the circular molecule comprises use of a ligase.

14. The method of claim 13, wherein the ligase is a DNA ligase or an RNA ligase.

15. The method of any one of claims 1-14, wherein forming the circular molecule comprises removal of a blocker molecule from the probe library.

16. The method of claim 15, wherein the blocker molecule comprises DNA, RNA, PNA, or LNA.

17. The method of any one of claims 1-16, wherein the restriction enzyme is a Type IIs restriction enzyme.

18. The method of any one of claims 1-16, wherein a first restriction enzyme is a Type II restriction enzyme and a second restriction enzyme is Type IIs restriction enzyme.

19. The method of claim 18, wherein cleavage with the Type II restriction enzyme and the Type IIs restriction enzyme is performed in a single step.

20. The method of any one of claims 17-19, wherein the Type IIs restriction enzyme cleaves 1 to 4 bases from the end of the target nucleic acid.

21. A method for sequencing of a target nucleic acid comprising:
(a) attaching a sample modifier comprising a moiety for immobilization to a solid support and a pre-specified sequence to a target nucleic acid to provide a modified nucleic acid;
(b) immobilizing the modified nucleic acid onto a solid support;
(c) ligating a probe library comprising a pre-determined expanded code for one or more bases to the immobilized modified nucleic acid;
(d) forming a circular molecule by circularizing the molecule produced in step (c);
(e) cleaving the circular molecule with one or more restriction enzymes;
(f) repeating steps (c) to (e) two or more times to provide a converted molecule;
(g) hybridizing the converted molecule to a plurality of detectably labeled molecules to form a complex;
(h) detaching the complex from the solid support; and
(i) translocating the complex through a nanopore,
wherein the translocation produces detectable signals which can be used to determine the nucleotide sequence of the target nucleic acid.

22. The method of claim 21, wherein the target nucleic acid is DNA or RNA.

23. The method of any one of claims 21-22, wherein the moiety for immobilization to a solid support is a biotin moiety.

24. The method of any one of claims 21-23, wherein the sample modifier is attached to the target nucleic acid using a ligase.

25. The method of claim 21, wherein the sample modifier comprises a barcode, a cleavage site, or a tag sequence.

26. The method of claim 25, wherein the cleavage site is a substrate for an enzyme or a chemical.

27. The method of claim 25, wherein the barcode identifies the sample of origin and is formed by the arrangement of the pre-determined expanded base codes forming a barcode 4-10 codes in length.

28. The method of claim 25, wherein the tag sequence identifies the 5′ end or the 3′ end of the converted molecule.

29. The method of any one of claims 20-28, wherein the solid support is a magnetic particle, polymeric microsphere, or a filter material.

30. The method of any one of claims 20-29, wherein the probe library comprises a plurality of distinct oligonucleotide sequences, each of which includes a double-stranded region, wherein the double-stranded region comprises: two restriction enzyme binding sites, the pre-specified nucleotide sequence, one or more pre-determined codes for each of the bases found in the target nucleic acid, and a first and a second single-stranded overhang, wherein the first single-stranded overhang is a complement to the pre-specified nucleotide sequence and the second single-stranded overhang comprises a plurality of sequences able to complement the target nucleic sequence.

31. The method of claim 30, wherein the pre-determined base codes bind to a molecular beacon.

32. The method of claim 31, wherein there are four pre-determined base codes.

33. The method of any one of claims 20-31, wherein forming the circular molecule comprises use of a ligase.

34. The method of claim 33, wherein the ligase is a DNA ligase or an RNA ligase.

35. The method of any one of claims 21-34, wherein forming the circular molecules comprises removal of a blocker molecule from the probe library.

36. The method of claim 35, wherein the blocker molecule comprises DNA, RNA, PNA, or LNA.

37. The method of any one of claims 21-37, wherein the restriction enzyme is a Type IIs restriction enzyme.

38. The method of any one of claims 21-36, wherein a first restriction enzyme is a Type II restriction enzyme and a second restriction enzyme is Type IIs restriction enzyme.

39. The method of claim 38, wherein the Type II and Type IIs restriction enzymes are combined together in a single step.

40. The method of any one of claims 37-39, wherein the Type IIs restriction enzyme cleaves 1 to 4 bases from the end of the target nucleic acid.

41. The method of any one of claims 21-40, wherein the detectably labeled molecules are optically detectable.

42. The method of claim 41, wherein the detectably labeled molecules comprise a fluorophore.

43. The method of claim 41, wherein the detectably labeled molecules comprise a fluorophore and a quencher.

44. The method of any one of claims 21-43, wherein the detectably labeled molecules comprise a bulky group.

45. The method of any one of claims 21-44, wherein the step of detaching from the solid support comprises using light, a chemical, or an enzyme.

46. The method of claim 45, wherein the enzyme is a restriction enzyme.

47. The method of claim 45, wherein the chemical is silver or periodate.
